(19) United States
(12) Patent Application Publication
(10) Pub. No.: US 2024/0245680 A1
(43) Pub. Date: Jul. 25, 2024

(54) Methods of Treating Retinal Vasculopathies

(71) Applicants: Scott KIMURA, South San Francisco, CA (US); Unity Biotechnology, Inc., Brisbane, CA (US)

(72) Inventors: Remi-Martin Laberge, Brisbane, CA (US); Scott Kimura, Brisbane, CA (US); Przemyslaw Sapieha, Brisbane, CA (US); Pamela Tsuruda, Brisbane, CA (US); Pieter Bas-Kwak, Brisbane, CA (US); Nathan Gushwa, Brisbane, CA (US); Tianna Chow, Brisbane, CA (US)

(21) Appl. No.: 18/289,554

(22) PCT Filed: May 25, 2022
(86) PCT No.: PCT/US2022/030938

§ 371 Date: Nov. 3, 2023

Related U.S. Application Data
(60) Provisional application No. 63/193,327, filed on May 26, 2021.
(60) Provisional application No. 63/220,771, filed on Jul. 12, 2021.

(51) Int. Cl. A61K 31/497; A61K 31/4152; A61K 31/437; A61K 45/06; A61P 27/02

(52) U.S. Cl. CPC A61K 31/497; A61K 31/4152; A61K 31/437; A61K 45/06; A61P 27/02

(57) ABSTRACT

The invention relates to methods of treating certain retinal vasculopathies such as diabetic macular edema, diabetic retinopathy and dry and neovascular age-related macular degeneration, among others. In some instances, the method includes treating a patient suffering from a retinal vasculopathy by administering an agent to the patient. The agent can be one or more of the agents as described herein.

### CROSS-REFERENCE TO RELATED APPLICATIONS

This application claims the benefit of U.S. Provisional Application No. 63/193,327, filed May 26, 2021, and U.S. Provisional Application No. 63/220,771, filed Jul. 12, 2021, the disclosures of each of which are incorporated herein by reference.

### FIELD OF THE INVENTION

The invention relates to methods of treating certain retinal vasculopathies such as diabetic macular edema, diabetic retinopathy and age-related macular degeneration.

### BACKGROUND OF THE INVENTION

Pathological angiogenesis remains one of the main challenges in the treatment of certain retinal vasculopathies such as diabetic macular edema (DME), diabetic retinopathy (DR) and age-related macular degeneration (AMD), which are severe diseases often leading to visual loss and blindness.

DME is a complication of diabetic retinopathy (DR) following chronic, poorly controlled diabetes, and is the most common form of sight-threatening retinopathy in people with diabetes (Tan et al. 2016, IDF 2019). Approximately one in 14 patients with diabetes has some degree of DME (Coney 2019). The overall prevalence of DR in patients with diabetes using retinal images was estimated to be 35%, with vision-threatening DR present in 12% (WHO 2015). Prevalence depends on the type of diabetes and the duration of the disease. For both types of diabetes, type 1 diabetes (T1D) and type 2 diabetes (T2D), after 25 years duration, prevalence approximates 30% (Browning et al. 2018).

Previous standards of care to stabilize vision in patients with such ocular diseases encompassed laser photocoagulation therapy, surgical vitrectomy and photodynamic therapy with verteporfin. Currently, anti-vascular endothelial growth factor (anti-VEGF) therapies and their ability to restore vision in patients suffering from such ocular vascular diseases has been widely used since the approval of Lucentis® (ranibizumab) in 2006 and Eylea® (aflibercept) in 2011. The recent success of treatments inhibiting the function of VEGF demonstrates that specific targeting of a growth factor responsible for vascular permeability and growth is an effective means of treating DR-associated vascular dysfunction, edema and angiogenesis.

However, such anti-VEGF treatments often led to off-target effects on healthy blood vessels. Therefore, discerning the molecular discrepancies between healthy and diseased blood vessels would allow for targeted treatments that selectively eliminate pathological vasculature while sparing vessels essential for physiological tissue function.

Another drawback to anti-VEGF standards of care is that they require frequent (e.g., monthly and even bimonthly intravitreal injections) and long-term administration to maintain vision gains (Heier et al. Ophthalmology 2012; 119:2537-48; the Comparison of Age-Related Macular Degeneration Treatment Trials [CATT] Research Group 2016 Ophthalmology 2016; 123:1751-61). This has led to poor patient compliance with this frequent treatment regimen.

There are also evidence of some patients experiencing suboptimal responses to anti-VEGF treatment as well as some patients that do not respond to anti-VEGF treatments at all. See Brown D M, et al., Ophthalmology. 2013; 120:2013-2022; and Nguyen-Khoa B A, et al., BMC Ophthalmol. 2012; 12:11.

Recently, the presence and accumulation of senescent cells in an individual may contribute to aging and aging-related dysfunction and diseases, such as, for example, glaucoma, cataracts, the diabetic pancreas and osteoarthritis, among others (see van Deursen J M., Nature. 2014 May 22; 509 (7501): 439-446; Childs, B. et al., Nat Med. 2015 December; 21(12): 1424-1435). Normally mitotic cells can permanently withdraw from the cell cycle in response to cellular stress, including dysfunctional telomeres, DNA damage, strong mitogenic signals, and disrupted chromatin. This response is termed cellular senescence and has been shown to be important to inhibiting proliferation of dysfunctional or damaged cells and particularly to constraining development of cancer malignancy mechanisms (see Campisi J., Cell 120:513-22 (2005); Campisi J., Curr. Opin. Genet. Dev. 21: 107-12 (2011)). Senescent cells are characterized by numerous cellular phenotypes, including insensitivity to mitogenic stimuli, flattened morphology, increased senescence-associated ß-galactosidase activity (SA-ß-gal), elevated p16 expression, shortened telomeres, elevated cyclin-dependent kinase inhibitor expression, changes in chromatin structure, pervasive DNA damage foci, resistance to apoptosis and activation of the pro-inflammatory senescence-associated secretory phenotype (SASP) (see Coppe, J-P, et al., Annu Rev Pathol. 2010; 5: 99-118).

It was recently shown that pathological vasculature in the retina selectively engages cellular senescence (see Crespo-Garcia et al., Cell Metabolism 2021, 33,1-15). Given that senescent cells have been causally implicated in certain aspects of age-related decline in health and likely contributes to certain age-related diseases, including cancer, effective therapeutics are being researched and developed. In some cases, small-molecule compounds have been identified that selectively remove accumulated senescent cells in and around the affected area, alleviating adverse signs and symptoms of the resulting conditions. Several intracellular pathways that are active in senescent cells have been shown to be amenable to targeting, such as, for example, the MDM2 pathway, the Bcl pathway, the Akt pathway, and the proteasome pathway, among others (see WO/2015/171591: Zhou et al.; WO/2015/116740: Laberge et al.; WO/2019/133904: Hudson et al.).

Aside from, or in addition to targeting senescent cells in the retina, an alternative mechanism whereby VEGF expression may be increased in retinal vasculopathies involves endoplasmic reticulum (ER) stress and the activating transcription factor 4 (ATF4). ATF4 is one of several transcription factors that are activated as part of the unfolded protein response (UPR) to stress. Translation of ATF4 mRNA into ATF4 protein is induced in response to several stresses that result in inhibition of general protein synthesis via phosphorylation of the eukaryotic translation initiation factor 2 (eIF2) (see Baird T D, et al., Adv Nutr. 2012; 3:307-321). These stresses include nutrient deprivation, ER stress, oxidative stress, and hypoxia (see Abcouwer, Steven F., J Clin Cell Immunol; Suppl 1(11):1-12, 2013). A decline in an adaptive response and hormesis with age to such stress provides an opportunity for treatments targeting this mechanism.

Therefore, a need exists for the development of alternative methods for treating retinal vasculopathies.

### SUMMARY OF THE INVENTION

The present invention provides novel methods of treating retinal vasculopathies. Specifically contemplated as part of the disclosed invention is:
Embodiment 1. A method of inhibiting pathogenic neovascularization in a diseased eye comprising administering an agent, wherein the agent is selected from the group consisting of an inhibitor of GPX4, an inhibitor of GLS1, an inhibitor of PAPP-A, an inhibitor of cGAS, an inhibitor of STING, an inhibitor of mTOR, or an agonist of GCN2.
Embodiment 2. A method of inhibiting vascular leak in a diseased eye comprising administering an agent, wherein the agent is selected from the group consisting of an inhibitor of GPX4, an inhibitor of GLS1, an inhibitor of PAPP-A, an inhibitor of cGAS, an inhibitor of STING, an inhibitor of mTOR, or an agonist of GCN2.
Embodiment 3. The method of any of embodiments 1 or 2, wherein the diseased eye is a result of senescent endothelial cells.
Embodiment 4. The method of any of embodiments 1 or 2, wherein the diseased eye is a result of senescent pericytes.
Embodiment 5. The method of any of embodiments 1 or 2, wherein the diseased eye is a result of diabetic macular edema (DME), diabetic retinopathy (DR), proliferative diabetic retinopathy (PDR), dry age-related macular degeneration (dAMD), and neovascular age-related macular degeneration (nvAMD).
Embodiment 6. A method of eliminating senescent endothelial cells comprising contacting the cells with an inhibitor of GPX4, an inhibitor of GLS1, an inhibitor of PAPP-A, an inhibitor of cGAS, an inhibitor of STING, an inhibitor of mTOR, or an agonist of GCN2.
Embodiment 7. A method of eliminating senescent pericytes comprising contacting the cells with an inhibitor of GPX4, an inhibitor of GLS1, an inhibitor of PAPP-A, an inhibitor of cGAS, an inhibitor of STING, an inhibitor of mTOR, or an agonist of GCN2.
Embodiment 8. A method of treating a patient suffering from a retinal vasculopathy comprising administering an agent to the patient, wherein the agent is selected from the group consisting of an inhibitor of GPX4, an inhibitor of GLS1, an inhibitor of PAPP-A, an inhibitor of cGAS, an inhibitor of STING, an inhibitor of mTOR, or an agonist of GCN2.
Embodiment 9. The method of embodiment 8, wherein the retinal vasculopathy is diabetic macular edema (DME), diabetic retinopathy (DR), proliferative diabetic retinopathy (PDR), dry age-related macular degeneration (dAMD), and neovascular age-related macular degeneration (nvAMD).
Embodiment 10. The method of any one of embodiments 1-5, 8-9, wherein the agent is a small molecule, an antibody, a polypeptide, an antisense oligonucleotide, a small-interfering ribonucleic acid.
Embodiment 11. The method of embodiment 6 or 7, wherein the inhibitor or agonist is a small molecule, an antibody, a polypeptide, an antisense oligonucleotide, a small-interfering ribonucleic acid.
Embodiment 12. The method of any one of embodiments 1-11, wherein the inhibitor of GPX4 is selected from the group consisting of RSL3, ML210, ML162, JKE-1674, DPI-7, buthionine sulfoximine (BSO), FIN56, auranofin, erastin, artemisinin, sulfasalazine, artesunate, dihydroatemisin, Compound #19, Compound #25, sorafenib, altretamine, almitrine, artemether, artemisone, lanperisone.
Embodiment 13. The method of any one of embodiments 1-11, wherein the inhibitor of GLS1 is 6-Diazo-5-oxo-L-norleucine, GK921, UPGL00004, telaglenastat, JHU395, Ethyl 2-(2-Amino-4-methylpentanamido)-DON, IPN60090, and BPTES.
Embodiment 14. The method of any one of embodiments 1-11, wherein the inhibitor of PAPP-A is an antibody.
Embodiment 15. The method of embodiment 14, wherein the antibody is PAC-1 scFV, PAC-1-D8 scFv, PAC-2 scFv, PAC-5 scFv, and mAb-PA 1/41.
Embodiment 16. The method of any one of embodiments 1-11, wherein the inhibitor of PAPP-A is a polypeptide.
Embodiment 17. The method of embodiment 16, wherein the polypeptide is pro-MBP, stanniocalcin-1 (STC1), stanniocalcin-2 (STC2) and bikunin.
Embodiment 18. The method of any one of claims 1-11, wherein the inhibitor of PAPP-A is an antisense oligonucleotide.
Embodiment 19. The method of any one of embodiments 1-11, wherein the inhibitor of cGAS is 3-(1-(6,7-dichloro-1H-benzo[d]imidazol-2-yl)-5-hydroxy-3-methyl-1H-pyrazol-4-yl)isobenzofuran-1(3H)-one, 1-(6,7-Dichloro-9-(1-methyl-1H-pyrazol-3-yl)-1,3,4,5-tetrahydro-2H-pyrido[4,3-b]indol-2-yl)-2 hydroxyethan-1-one, 1-[9-(6-amino-3-pyridinyl)-6,7-dichloro-1,3,4,5-tetrahydro-2H-pyrido[4,3b]indol-2-yl]-2-hydroxy-ethanone, or (1R,2S)-2-(7-Oxo-5-phenyl-4,7-dihydropyrazolo[1,5-a]pyrimidine-3 carboxamido) cyclohexane-1-carboxylic acid.
Embodiment 20. The method of any one of embodiments 1-11, wherein the inhibitor of STING is H-151, C-178, C-176, and Compound 18.
Embodiment 21. The method of any one of embodiments 1-11, wherein the inhibitor of mTOR is rapamycin, Palomid 529, sirolimus, everolimus, temsirolimus, ridaforolimus, umirolimus, zotarolimus and ATP-competitive mTOR kinase inhibitors.
Embodiment 22. The method of any one of embodiments 1-11, wherein the agonist of GCN2 is 4-(2-amino-4-methyl-3-(2-methylquinolin-6-yl)benzoyl)-1-methyl-2,5-diphenyl-1H-pyrazol-3(2H)-one, 1-(5-(4-amino-2,7-dimethyl-7H-pyrrolo[2,3-d]pyrimidin-5-yl)indolin-1-yl)-2-(3-fluoro-5-(trifluoromethyl)phenyl)-ethanone, 4-(2-Amino-4-methyl-3-(2-(methylamino)benzo[d]thiazol-6-yl)benzoyl)-1-methyl-2,5-diphenyl-1H-pyrazol-3(2H)-one, leucenol, histidinol, threoninol, SB-203207, SB-219383, dovitinib, neratinib, sunitinib, and elotinib.

### BRIEF DESCRIPTION OF THE DRAWINGS

FIG. 1 depicts the quantification of senescent cell burden in AMD and DR/DME as assessed by immunohistochemistry staining for p16 as compared to normal in post-mortem retinal donor tissues from patients who carried a pre-mortem diagnosis of AMD, DR/DME or neither. See Example 2.

FIG. 2 depicts the quantification of senescent cell burden in PDR as assessed by immunohistochemistry staining for p16 as compared to normal in post-mortem retinal donor tissues from patients who carried a pre-mortem diagnosis of PDR or neither. See Example 2.

FIGS. 3A-3B depicts a GPX4 inhibitor, RSL3, that demonstrates selective killing of irradiated senescent HRMEC cells over non-senescent HRMEC cells in a dose-dependent manner (FIG. 3A) and of irradiated senescent HUVEC cells over non-senescent HUVEC cells in a dose-dependent manner (FIG. 3B), see Example 1.

FIG. 4 depicts a GPX4 inhibitor, JKE-1674, that demonstrates selective killing of irradiated senescent HUVEC cells over non-senescent HUVEC cells in a dose-dependent manner, see Example 1.

FIG. 5 depicts a GPX4 inhibitor, ML210, that demonstrates selective killing of irradiated senescent HRMEC cells over non-senescent HRMEC cells in a dose-dependent manner, see Example 1.

FIGS. 6A and 6B both depict two GPX4 inhibitors, Compound #19 (FIG. 6A) and Compound #25 (FIG. 6B) demonstrating selective elimination of irradiated HUVEC cells over non-senescent HUVEC cells in a dose-dependent manner, see Example 1.

FIG. 7 is a schematic of a GPX4 competitive target engagement gel-shift assay used to detect inhibition of GPX4 in cell lines or in tissues, see Example 7.

FIGS. 8A-8C depicts target engagement of a GPX4 inhibitor, RSL3, in adult murine retinas. A gel shift assay was performed where each lane contains 20 μL of retinal lysate labeled with RSL3 and streptavidin for the gel shift (FIG. 8A). For quality control, equal parts vehicle treated retinal lysates were combined and treated with 100 μM RSL3 or DMSO (FIG. 8B). Target engagement was quantified based on an integrated band density measurement (ImageJ®) (FIG. 8C). See Example 7.

FIGS. 9A-9B depicts the target engagement of GPX4 inhibitors Compounds #19 (FIG. 9A) and #25 (FIG. 9B) by incubation with senescent HUVEC cells for 6 hours. See Example 7.

FIGS. 10A-10D depicts GCN2 pathway activation using the reporter genes ATF4 (FIGS. 10A and 10B) and DDIT3 (FIGS. 10C and 10D) in H2122 cells. See Example 8.

FIGS. 11A-11C depicts that a systemic administration of a GCN2 activator, Compound 39, activates GCN2 in the retina. FIGS. 11A and 11B show that Compound 39 activated reporter genes ATF4 and DDIT3 in the retina at various doses and timepoints in both normoxic and oxygen-induced retinopathy (OIR) mice. FIG. 11C shows that intraperitoneally administered Compound 39 activated ATF4 protein expression at 30 and 60 mpk in the mouse retina. See Example 9.

FIGS. 12A and 12B shows that a GCN2 activator, Compound 39, regulates disease-relevant gene expression by activating the expression of both Slc7a11, a redox factor (FIG. 12A), and Serpinf1, an anti-angiogenic factor (FIG. 12B) following intraperitoneal administration at various doses and timepoints in both normoxic and OIR neonatal mice. See Example 10.

FIG. 13 shows that intraperitoneally administered Compound 39, a GCN2 activator, at 30 mpk activated ATF4 protein expression, increased phosphor-eIF2a levels and downregulated HIF1α protein expression as early as 4 hours post-treatment in retinas from OIR neonates. See Example 10.

FIGS. 14A-14B show that GCN2 activation improves the retinal vasculature in the OIR mouse model. Specifically Compound 39, a GCN2 activator, when systemically administered can improve neovascularization (FIG. 14A) but not avascular area (FIG. 14B) in the retinas of treated OIR mice. See Example 11.

FIGS. 15A-15B show that GCN2 activation improves neovascularization in the OIR mouse model. Compound 39 can improve neovascularization (FIG. 15B) in the retinas of treated OIR mice as compared to a vehicle control (FIG. 15A). See Example 11.

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) embodiments that are directed to that value or parameter per se.

As used herein, “administering” is means a method of giving a dosage of the Compound-meglumine to a patient suffering from the retinal vasculopathies herein. The compositions utilized in the methods described herein can be administered, for example, intravitreally (e.g., by intravitreal injection), ocularly (e.g., by ocular injection), or intraocularly (e.g., by intraocular injection). The method of administration can vary depending on various factors (e.g., the compound or composition being administered, and the severity of the condition, disease, or disorder being treated).

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

As used herein, the term “anti-VEGF treatment” or “anti-VEGF therapy” means any currently approved anti-VEGF drugs indicated to treat retinal vasculopathies. Such approved drugs include, for example, Lucentis® (ranibizumab) or Eylea® (aflibercept) and any approved biosimilar drugs to ranibizumab or aflibercept.

An “agonistic” or “activator” or “activating” agent is one which activates, stimulates or increases biological or signaling activity of the target or antigen it binds. In some situations, it is contemplated that an agonistic agent can act in a similar manner to how a ligand engages and activates its cognate receptor.

As used herein, “ferroptosis” refers to a form of cell death understood in the art as involving generation of reactive oxygen species mediated by iron, and characterized by, in part, lipid peroxidation. “Ferroptosis inducer” or “ferroptosis activator” refers to an agent which induces, promotes or activates ferroptosis.

As used herein, “retinal vasculopathies” refers to ocular diseases that involve pathogenic angiogenesis, such as, for example, diabetic macular edema, diabetic retinopathy and dry or neovascular (or wet) age-related macular degeneration. Other ocular diseases can include, for example, geographic atrophy.

A “senescent cell” is generally thought to be derived from a cell type that typically replicates, but as a result of aging or other event that causes a change in cell state, no longer replicates. In other words, a senescent cell is one that enters into an irreversible arrest of cell proliferation. A senescent cell also becomes resistant to apoptosis and therefore persists in a subject and can accumulate as the subject ages. Further, a senescent cell exhibits a multi-faceted secretory phenotype or senescence associated secretory phenotype (SASP), which are believed to be pathological soluble factors that can play a role in the initial presentation or ongoing pathology of a senescence-associated cancer. For the purpose of practicing aspects of this invention, senescent cells may be identified as, for example, expressing p16, or at least one marker selected from p16, senescence-associated ß galactosidase, or p21. A senescent cell may also be identified as that which secretes SASP factors such as, but not limited to, inflammatory factors (such as, for example, but not limited to MMP1, MMP3, TNF-α, IL-1β, prostaglandins), and/or pro-fibrotic factors (such as, for example, but not limited to TGFβ1, TGFβ2, CTGF, TIMP-1, MCP-1) and/or growth factors (such as, for example, but not limited to VEGF-α, IL-6, IL-8, Pai-1).

A candidate agent inhibitor or agonist is typically referred to as “senolytic” if it eliminates senescent cells more selectively as compared with replicative cells of the same tissue type, or quiescent cells lacking SASP markers. In one aspect, achieving about a 2-10 fold selectivity, or about a 10-100 fold selectivity, or about a 10-1000 fold selectivity, or about a 100-2000 fold selectivity, or about a 10-2000 fold selectivity, or about a 1000-2000 fold selectivity, or greater than 1000 fold selectivity for the elimination of a senescent cell over a non-senescent cell is contemplated as part of the methods of the invention. Alternatively, or in addition, the methods of the invention may effectively be used if it decreases the release of pathological soluble factors or mediators as part of the senescence associated secretory phenotype (SASP) that play a role in the initial presentation or ongoing pathology of a condition or inhibit its resolution. In this respect, the term “senolytic” is exemplary, with the understanding that the compounds and methods of the invention may work primarily by selectively inhibiting, rather than eliminating, senescent cells (senescent cell inhibitors) and such can be used in the methods of the invention with ensuing benefits.

As used herein, “treatment” (and “treat” or “treating”) refers to clinical intervention in an attempt to alter the natural course of the individual being treated and can be performed either for prophylaxis or during the course of clinical pathology. Desirable effects of the methods of treatment as claimed herein include, but are not limited to, preventing occurrence or recurrence of DME, DR or AMD, alleviation of symptoms of DME, DR or AMD, diminishment of any direct or indirect pathological consequences of DME, DR or AMD, decreasing the rate of disease progression in DME, DR or AMD, amelioration or palliation of the disease state in DME, DR or AMD, and remission or improved prognosis for DME, DR or AMD.

Except where otherwise stated or required, other terms used in the specification have their ordinary meaning.

### Compositions and Methods

The invention provides methods for treating certain retinal vasculopathies, such as diabetic macular edema (DME), diabetic retinopathy (DR), and age-related macular degeneration (AMD).

DME is a complication of diabetic retinopathy (DR) following chronic, poorly controlled diabetes, and is the most common form of sight-threatening retinopathy in people with diabetes. Approximately one in 14 patients with diabetes has some degree of DME. The World Health Organization reported in 2015 that the overall prevalence of DR in patients with diabetes using retinal images was estimated to be 35%, with vision-threatening DR present in 12%. Prevalence depends on the type of diabetes and the duration of the disease. For both types of diabetes, type 1 diabetes and type 2 diabetes, after 25 years duration, prevalence approximates 30%. In the US, at least 5.5 million individuals over the age of 40 are estimated to have DR in the absence of DME, and an additional 800,000 to 1 million patients have DME. According to some estimates, only 40% of them diagnosed and treated, and about 5% are diagnosed and observed.

Diabetic macular edema (DME), a macular thickening secondary to diabetic retinopathy (DR), results from a blood-retinal barrier defect that leads to vascular leakage and fluid accumulation. See Bhagat N, et al., Surv Ophthalmol. 2009; 54:1-32. DME has been related to the expression of several inflammatory factors, including vascular endothelial growth factor (VEGF), intercellular adhesion molecule-1 (ICAM-1), interleukin-6 (IL-6), monocyte chemotactic protein-1 (MCP-1), and leukostasis. See Funatsu H, et al., Ophthalmology. 2009; 116:73-79; and Miyamoto K, et al., Proc Natl Acad Sci USA. 1999; 96:10836-10841. Moreover, the expression of these factors has been related to both vascular permeability of the retina along with the severity of disease, thus confirming their important pathogenetic role.

DR: The prevalence of diabetic retinopathy increases with age and is the most common cause of blindness in people over the age of 50. It is a multifactorial disorder, with hyperglycemia exerting toxic effects on cells and inflammatory cytokine implicated in many aspects of diabetic eye disease. Diabetic retinopathy involves thickening of capillary basement membranes and prevents pericytes from contacting endothelial cells of the capillaries. Loss of pericytes increases leakage of the capillaries and can lead to a breakdown of the blood-retina barrier. Weakened capillaries can lead to aneurysm formation and further leakage. The duration and severity of hyperglycemia is a factor linked to the development of diabetic retinopathy. These effects of hyperglycemia can also impair neuronal functions in the retina. This is an early stage of diabetic retinopathy termed nonproliferative diabetic retinopathy (NPDR).

Diabetic retinopathy is also a degenerative disease of the neural retina, associated with alterations in neuronal function prior to the onset of clinical vascular disease. Retinal capillaries can become occluded in diabetes causing areas of ischemia in the retina. The non-perfused tissue responds by eliciting new blood vessel growth from existing vessels (i.e., angiogenesis). These new blood vessels can also cause loss of sight, a condition called proliferative diabetic retinopathy (PDR), since the new blood vessels are fragile and tend to leak blood into the eye. In advanced proliferative diabetic retinopathy, an angiogenic, VEGF-mediated response with retinal neovascularization ensues, placing the eye at further risk for severe visual loss due to the development of vitreous hemorrhage or traction retinal detachment. Irreversible vascular or neuronal damage is possible without treatment, underscoring the need for early intervention.

AMD: Age-related macular degeneration (AMD) is a leading cause of visual impairment and severe vision loss. It ranks third among the global causes of visual impairment with a blindness prevalence of 8.7%.

AMD is caused by the deterioration of the central portion of the retina and is a multifactorial disorder, with dysregulation in the complement, lipid, angiogenic, inflammatory, and extracellular matrix pathways implicated in its pathogenesis. AMD pathology is characterized by a progressive accumulation of characteristic yellow deposits, called drusen (e.g., a buildup of extracellular proteins and lipids), in the macula, between the retinal pigment epithelium and the underlying choroid which is believed to damage the retina over time.

There are two basic types of AMD: “dry” and “wet” (also known as neovascular AMD or nvAMD). Approximately 85% to 90% of the cases of AMD are the “dry” (atrophic) type, while 10-15% are the “wet” (exudative) type. Dry AMD (dAMD) is defined by the gradual loss of retinal pigment epithelial (RPE) and photoreceptor cells in the macula. Patients who are affected by dry AMD have gradual loss of central vision due to the death of photoreceptor cells and their close associates, retinal pigmented epithelial (RPE) cells, with deposition of drusen. Wet AMD is characterized by the growth of abnormal blood vessels beneath the macular epithelium. Wet AMD results in vision loss due to abnormal blood vessel growth (e.g., choroidal neovascularization) in the choriocapillaris, through Bruch's membrane. Clinically, it is classified as early-stage (medium-sized drusen and retinal pigmentary changes) to late-stage (neovascular AMD and atrophic AMD).

Senolytic Assays: Candidate agents of the invention can also be evaluated for an ability to kill senescent cells selectively. Cultured cells are contacted with the agent, and the degree of cytotoxicity or inhibition of the cells is determined. The ability of the agent to kill or inhibit senescent cells can be compared with the effect of the agent on normal cells that are freely dividing at low density, and normal cells that are in a quiescent state at high density. As a non-limiting example, Example 1 provides illustrations of cell-based assays using irradiation-induced senescence in the human endothelial HUVEC cell line and the human RPE cell line to test and quantify candidate agents of the invention. Similar protocols are known and can be developed or optimized for testing the ability of candidate senolytic compounds to kill other senescent cell types.

Pathways: The invention described and claimed herein contemplates using inhibitors or agonists of certain identified biological pathways to treat retinal vasculopathies. The invention described and claimed herein also contemplates using inhibitors or agonists of certain identified biological pathways to eliminate senescent cells, such as senescent endothelial cells and/or senescent pericytes, to treat retinal vasculopathies. The contemplated biological pathways are as follows.

Glutathione peroxidase 4 (GPX4) pathway: Regulation of oxidative stress and redox systems has important roles in carcinogenesis and cancer progression, and for this reason has attracted much attention as an area of cancer therapeutic targets. Glutathione peroxidase 4 (GPX4) belongs to the family of glutathione peroxidases, which consists of 8 known mammalian isoenzymes (GPX1-8), members of which catalyze the reduction of hydrogen peroxide, organic hydroperoxides and lipid hydroperoxides, and thereby protect cells against oxidative stress and will signal cell death by suppressing peroxidation of membrane phospholipids. When GPX4 activity is compromised, lipid peroxidation can cause ferroptosis, an oxidative iron-dependent form of non-apoptotic cell death. Ferroptosis induced by exogenous agents is selectively lethal towards tumors cells that are addicted to GPX4 repair activity, which has suggested that inducing ferroptosis may be a beneficial approach to treating some cancers. See Stockwell B R, et al., Cell. 2017; 171:273-285.

Exemplary GPX4 Inhibitors: Any GPX4 inhibitor currently known in the art or to be developed can be tested for senolytic activity and developed for treatment of senescence-associated and/or age-related diseases in accordance with this invention. In particular, GPX4 inhibitors that can be utilized in the methods of the invention include, without limitation, for example, RSL3, ML210, ML162, JKE-1674, DPI-7, buthionine sulfoximine (BSO), FIN56, auranofin, erastin, artemisinin, sulfasalazine, artesunate, dihydroatemisin, sorafenib, altretamine, almitrine, artemether, artemisone, or lanperisone. Other non-limiting exemplary GPX4 inhibitors can be Compounds 1-273 as disclosed and claimed in WO 2019/168999 and Compounds 1-102 as disclosed and claimed in WO 2020/176757 and Compounds 1-12 and Compounds A-1 to A-19 as disclosed and claimed in WO 2021/041536, each of which is incorporated by reference in each of their entireties.

GPX4 Biological Assays: Studies have shown that lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, the ability of a molecule to induce ferroptotic cancer cell death, and that such ability is admonished by the addition of ferrostatin, is clear indication that the molecule is an GPX4 inhibitor. Exemplary assays used to test the inhibition potential of candidate agent GPX4 inhibitors are as follows. A mobility shift of GPX4 Western blot assay can be used to assess target engagement directly in cell-based assay after incubation with candidate GPX4 inhibitor agents. Mobility shift can be used as a pharmacodynamic marker for GPX4 irreversible inhibitor agents. Further, candidate GPX4 inhibitor agents can be evaluated in cell-based Western blot analysis of GPX4.

Glutaminase inhibitor 1 (GLS1) pathway: Glutamine is the most abundant plasma amino acid and is involved in many growth promoting pathways. In particular, glutamine is involved in oxidation in the TCA cycle and in maintaining cell redox equilibrium, and also provides nitrogen for nucleotide and amino acid synthesis (Curi et al., Front. Biosci. 2007, 12, 344-57; DeBerardinis and Cheng, Oncogene 2010, 313-324). Glutaminase is a mitochondrial amidohydrolase enzyme that generates glutamate from glutamine. It has a role in the detoxification of ROS.

Exemplary GLS1 Inhibitors: Any GLS1 inhibitor currently known in the art or to be developed can be tested and developed for treatment of the disclosed retinal vasculopathies in accordance with this invention. In particular, GLS1 inhibitors that can be utilized in the methods of the invention include, without limitation, for example, 6-Diazo-5-oxo-L-norleucine (Diazooxonorleucine, L-6-Diazo-5-oxonorleucine, DON), an antibiotic isolated from Streptomyces, is a glutaminase antagonist with IC50 of ˜1 mM for cKGA (kidney-type glutaminase), GK921 is a transglutaminase 2 (TGase 2) inhibitor with an IC50 of 8.93 μM under a modified assay condition, UPGL00004 is a potent glutaminase C (GAC) inhibitor with an IC50 of 29 nM, showing high selectivity for GAC over GLS2, bis-2-(5-phenylacetamido-1, 2, 4-thiadiazol-2-yl) ethyl sulfide 3 (BPTES) is a potent and selective glutaminase GLS1 inhibitor with IC50 of 0.16 μM, Telaglenastat (CB-839) is a potent, selective, and orally bioavailable glutaminase inhibitor with IC50 of 24 nM for recombinant human GAC, JHU395 is an orally bioavailable GA (glutamine antagonists) prodrug designed to circulate inert in plasma, but permeate and release active GA within target tissues, JHU-083 (Ethyl 2-(2-Amino-4-methylpentanamido)-DON) is a novel prodrug of DON and selectively blocks glutaminase activity, IPN-60090 dihydrochloride (also known as IACS-6274) is an orally active and highly selective inhibitor of glutaminase 1 (GLS1; IC50=31 nM), with no activity observed against GLS-2.

GLS1 Biological Assays: Exemplary assays used to test the inhibition potential of candidate agent GLS1 inhibitors are as follows. A glutamate oxidase/AmplexRed® coupled assay can be used to measure the ability of GLS1 inhibitor compounds to bind to and inhibit the activity of purified, recombinant 6His tagged GLS1 in vitro. The binding reaction can be stopped by addition of the reagent 6-(2-bromoethynyl)-2,3-dimethyl-quinazolin-4-one, Amplex Red, Horseradish Peroxidase, and Glutamate Oxidase in a TRIS buffer. Reactions can be read on a Perkin Elmer EnVision® using 535/590 nm optic filters and raw data can be analysed using Genedata® to generate IC50 values. Another assay that can be used is a PC3 cell-coupled assay measuring cellular glutamate depletion using AmplexRed. PC3 cells can be seeded into multi-well plates containing candidate agent inhibitors. After incubation, plates can be read on a Perkin Elmer EnVision® using 535/590 nm optic filters and raw data analysed using proprietary software to generate IC50 values.

Pregnancy-associated plasma protein-A (PAPP-A) or Pappalysin-1 Pathway: PAPP-A, a zinc metalloproteinase, binds surface glycoaminoglycans (GAGs) and cleaves IGFBP-2, -4, and -5 to release IGF1 or IGF2 for local signaling. PAPP-A in pregnancy serum is linked via a disulfide bond to the proform of eosinophil major basic protein (proMBP), forming an approximately 500 kDa 2:2 complex, denoted PAPP-A/proMBP. The serum form of PAPP-A is derived from a preproprotein containing a putative 22-residue signal peptide, a pro-part of 58 residues, and a 1547-residue circulating mature polypeptide. The amino acid sequence shows no global similarity to any known protein, but it contains two sequence motifs common to the metzincins, a superfamily of metalloproteases, three Lin-12/Notch repeats known from the Notch protein superfamily, and five short consensus repeats known from components of the complement system. Transgenic knock-out of PAPP-A may result in lifespan extension in mice presumably by reducing circulating IGF-I levels (see Hotzenberger et al., Nature (2002) December 4 and Conover et al., Development, 131(5):1187-1194 (2004)).

Exemplary PAPP-A inhibitors: Any PAPP-A inhibitor currently known in the art or to be developed can be tested and developed for treatment of the disclosed retinal vasculopathies in accordance with this invention. In particular, PAPP-A inhibitors that can be utilized in the methods of the invention include, without limitation, for example, antibodies that specifically bind to PAPP-A. These can include, for example, the PAC-1 ScFV, PAC-1-D8 scFv, PAC-2 scFv, PAC-5 scFv, a humanized mAb-PA 1/41 as disclosed in WO 2020/198166, in U.S. Pat. No. 8,653,020, in Mikkelsen et al., Oncotarget 5, 1014-1025 (2014), in Mikkelsen et al., J. Biol. Chem. 283, 16772-16780 (2008), and in Mohrin et al., bioRxiv, doi: https://doi.org/10.1101/2020.02.05.936310; this version posted Feb. 6, 2020, all of which are all incorporated herein by reference, each in their entireties. Other PAPP-A inhibitors that can be utilized in the methods of the invention include, without limitation, for example, polypeptides that can inhibit the function of PAPP-A. These can include, for example, pro-MBP, stanniocalcin-1 (STC1), or stanniocalcin-2 (STC2). Still other PAPP-A inhibitors that can be utilized in the methods of the invention include, without limitation, for example, antisense oligonucleotides targeting PAPP-A such as the 18-base phosphorothioate oligodeoxynucleotides corresponding to the human PAPP-A mRNA consisting of the antisense sequences of 5′-GCCCAACTCCTGCTGGAA-3′ (AS-PAPP-A) (see Tanaka et al., Cancer Cell Biol January 2004). Still other PAPP-A inhibitors that can be utilized in the methods of the invention include, without limitation, the Kunitz-type protease inhibitor bikunin.

PAPP-A Biological Assays: Exemplary assays used to test the inhibition potential of candidate agent PAPP-A inhibitors are as follows. A cellular pAKT assay can be employed where IGFBP-4 protein is mixed with IGF1 and incubated for 30 min at 37° C. PAPP-A protein is added to IGFBP-4/IGF1 mix and incubated for 4-5 hours at 37° C. HEK293 cells can be plated in EMEM media without serum and allowed to adhere overnight. IGF1/IGFBP-4/PAPP-A mix is added to cells at 1:30 final dilution and incubated for 20 min at 37° C. Cells are lysed in MSD Tris Lysis buffer and analyzed by Phospho(Ser473)/Total Akt Whole Cell Lysate kit (MSD, K15100D) according to manufacturer's protocol. To evaluate neutralizing potency of an anti-PAPP A antibody, PAPP-A protein can be pre-incubated with various concentrations of an anti-PAPP-A antibody prior to adding to IGF1/IGFBP-4 mix.

Another PAPP-A biological assay that may be used is an in vitro PAPP-A enzymatic cleavage of IGF binding proteins. In such as assay, human and murine PAPP-A proteins with C-terminal C-myc and Flag tags are expressed by stably transduced HEK293 cell line and purified by heparin column chromatography. Human and murine IGFBP-2, IGFBP-4 and IGFBP-5 proteins are produced recombinantly by transient expression in HEK293 cells with either N-terminal (for human proteins) or C-terminal (for murine proteins) 6His tag and purified by Ni-Sepharose column chromatography. For enzymatic cleavage reaction, IGFBP-2 and IGFBP-4 proteins are pre-incubated with IGF1 of appropriate species (R&D Systems, 291-G1-200 for human and 791-MG-050 for mouse) for 30 min at 37° C. IGFBP 2/IGF1, IGFBP-4/IGF1 or IGFBP-5 proteins are then mixed with various concentrations of PAPP-A and incubated for 2-4 hours at 37° C. Final concentration in cleavage reactions can be 90 nM for IGFBPs and 850 nM for IGF1. Proteins are then resolved by capillary electrophoresis on Wes instrument (ProteinSimple) using capillary cartridge kit (ProteinSimple, cat. #SM-W002-1), probed with THE HisTag antibody (GeneScript, cat. #A00186) and visualized with anti-mouse detection module (ProteinSimple, cat. #DM-002). To evaluate neutralizing potency of anti-PAPP A antibody, PAPP-A protein can be pre-incubated with various concentrations of anti-PAPP-A antibody prior to adding to IGF1/IGFBP-4 mix. PAPP-A concentration is these assays can be fixed to 0.4 nM for IGFBP-4, 3.5 nM for IGFBP-2 and 0.08 nM for IGFBP-5 cleavage.

cGAS-STING Pathway: The recognition of microbial nucleic acids is a major mechanism by which the immune system detects pathogens. Cyclic GMP-AMP (cGAMP) synthase (cGAS) is a cytosolic DNA sensor that activates innate immune responses through production of the second messenger cGAMP, which activates the adaptor stimulator of interferon genes or STING. STING then recruits TANK-binding kinase 1 (TBK1) and IκB kinase to activate IFN regulatory factor 3 (IRF3) and NF-κB, respectively, leading to the production of type I interferons and inflammatory cytokines, which may also lead to autoimmune and inflammatory disease. cGAS has also been reported to be involved in cellular senescence, where mouse embryonic fibroblasts (MEFs) from cGas−/− mice displayed reduced signs of senescence and underwent faster spontaneous immortalization compared with MEFs from WT mice (see Yang et al., PNAS June 2017).

Exemplary cGAS Inhibitors: Any cGAS inhibitor currently known in the art or to be developed can be tested and developed for treatment of the disclosed retinal vasculopathies in accordance with this invention. In particular, cGAS inhibitors that can be utilized in the methods of the invention include, without limitation, for example, RU.521 which is 3-(1-(6,7-dichloro-1H-benzo[d]imidazol-2-yl)-5-hydroxy-3-methyl-1H-pyrazol-4-yl)isobenzofuran-1(3H)-one (see Vincent J. et al., 2017. Nat Commun. 28(1):750); or G140 which is 1-(6,7-Dichloro-9-(1-methyl-1H-pyrazol-3-yl)-1,3,4,5-tetrahydro-2H-pyrido[4,3-b]indol-2-yl)-2 hydroxyethan-1-one or G150 which is 1-[9-(6-amino-3-pyridinyl)-6,7-dichloro-1,3,4,5-tetrahydro-2H-pyrido[4,3b]indol-2-yl]-2-hydroxy-ethanone (for both G140 and G150 see Lama, L. et al. 2019. Nat Commun. 10, 2261); or PF-06928215 which is (1R,2S)-2-(7-Oxo-5-phenyl-4,7-dihydropyrazolo[1,5-a]pyrimidine-3 carboxamido) cyclohexane-1-carboxylic acid (see Hall et al., PLOS ONE 2017, 12, e0184843).

Exemplary STING Inhibitors: Any STING inhibitor currently known in the art or to be developed can be tested and developed for treatment of the disclosed retinal vasculopathies in accordance with this invention. In particular, STING inhibitors that can be utilized in the methods of the invention include, without limitation, for example, H-151 which is CAS No. 941987-60-6, or C-178 which is CAS No. 329198-87-0, or C-176 which is CAS No. 314054-00-7 (for H-151, C-178 and C-176 see Haag S. M. et al., 2018. Nature 559:269-73). Other STING antagonists are Compound 18 or C18 (see Siu, et al. ACS Med. Chem. Lett., 10 (1) (2019), pp. 92-97).

cGAS or STING Biological Assays: Exemplary assays used to test the inhibition potential of candidate agents of cGAS and/or STING are as follows. cGAS inhibitors can be measured for cGAS activity using a mass spectrometry assay by reactions with ATP, GTP, double-stranded DNA, and CGAS. Other cGAS activity assays include Cy5-cGAMP fluorescence polarization assay in which the candidate cGAS inhibitor agents are reacted with ATP or GTP or double-stranded DNA and cGAS. Cy5-labeled cGAMP and cGAMP antibody are added and the reactions are read on an Envision plate reader at 620 nm to determine the Ki (see Hall et al., PLOS ONE 2017, 12, e0184843). Other assays that may be utilized is a soluble adenylyl cyclase (sAC) inhibition assay in which candidate cGAS inhibitor agents are incubated with human sACt protein then ATP is added. Formation of CAMP and consumption of ATP is compared to standards using RF-MS and an IC50 is calculated.

mTOR Pathway: Mechanistic target of rapamycin (mTOR) is a highly conserved serine/threonine kinase that controls cell growth and metabolism in response to nutrients, growth factors, cellular energy, and stress.

Exemplary mTOR inhibitors: Any mTOR inhibitor currently known in the art or to be developed can be tested and developed for treatment of the disclosed retinal vasculopathies in accordance with this invention. In particular, STING inhibitors that can be utilized in the methods of the invention include, without limitation, for example, rapamycin, Palomid 529, sirolimus, everolimus, temsirolimus, ridaforolimus, umirolimus, zotarolimus and ATP-competitive mTOR kinase inhibitors.

General Control Nonderepressible 2 (GCN2) Pathway: Cells must be able to sense and adapt to their surroundings to thrive. Key to adapting to a low nutrient environment is the Integrated Stress Response (ISR), a pathway that allows cells to either regain cellular homeostasis or facilitate apoptosis during periods of stress. Central to the ISR is the serine/threonine protein kinase GCN2, which is responsible for sensing starvation. Upon amino acid deficiency, GCN2 is activated and initiates the ISR by phosphorylating the translation initiation factor eIF2a, stalling protein translation, and activating the transcription factor ATF4, which in turn up-regulates autophagy and biosynthesis pathways.

Exemplary GCN2 activators: Any GCN2 activator currently known in the art or to be developed can be tested and developed for treatment of the disclosed retinal vasculopathies in accordance with this invention. In particular, agonists of GCN2 that can be utilized in the methods of the invention include, without limitation, for example, 4-(2-amino-4-methyl-3-(2-methylquinolin-6-yl)benzoyl)-1-methyl-2,5-diphenyl-1H-pyrazol-3(2H)-one Hydrochloride or Compound 44, 1-(5-(4-amino-2,7-dimethyl-7H-pyrrolo[2,3-d]pyrimidin-5-yl)indolin-1-yl)-2-(3-fluoro-5-(trifluoromethyl)phenyl)-ethanone or Compound 52, 4-(2-Amino-4-methyl-3-(2-(methylamino)benzo[d]thiazol-6-yl)benzoyl)-1-methyl-2,5-diphenyl-1H-pyrazol-3(2H)-one or Compound 39 (see Smith, Adrian L., et al., J of Med Chem. 2015 Feb. 12:58(3):1426-41 for Compounds 39, 44 and 52), leucenol, histidinol, threoninol, SB-203207, SB-219383 (see Cavener, D., et al., WO 2008/085921), dovitinib, neratinib, sunitinib, and elotinib.

GCN2 Biological Assays: Exemplary assays used to test the activation potential of candidate agents of GCN2 can be, without limitation, as follows. One such assay is an eIF2a phosphorylation assay in which reactions combining GCN2, eIF2a and candidate GCN2 activator agents are performed upon the addition of ATP. The reactions are quenched and then denatured by boiling, then run on a protein gel, transferred to nitrocellulose or a PVDF membrane, blocked and incubated with an antibody against phosphor-eIF2a and then visualized after incubation with a chemiluminescent substrate. A cellular assay that may be used to test candidate GCN2 activators is to use a stable cell line, such as, for example, HEK293T cells transfected with an ATF4 reporter fused to a firefly luciferase (FLuc) to produce recombinant retroviruses. Such cells are plated and then treated with an appropriate amount of a candidate GCN2 activator agent for a period of time. Luminescence is measured and expressed as an EC50.

Methods of monitoring and diagnosing retinal vasculopathies: Standardized ophthalmic examination techniques known in the art include, for example, a detailed slit lamp biomicroscopic evaluation which allows evaluation of the lids, ocular adnexa, lashes, corneal surface, anterior chamber, pupils, lens, vitreous cavity and central retinal anatomy including the optic nerve and macula. Another method is gonioscopy which allows detailed examination of the anterior chamber angle. Indirect ophthalmoscopy allows for evaluation of the retinal periphery, important in the monitoring of vitreous and peripheral retinal disorders.

Functional tests of visual acuity are known in the art and include, for example, best corrected acuity, contrast acuity, and low luminance acuity, color vision (including Ishihara and Farnsworth Munsell tests) and visual field evaluation (including Humphrey automated perimetry and microperimetry), tear production (Schirmer test), and tonometry to measure intraocular pressure (IOP). These are used in conjunction with structural tests, which include, for example, anterior and posterior segment photographs, corneal pachymetry, ultrasound, ultrasound biomicroscopy, optical coherence tomography (OCT), optical coherence tomography angiography (OCTA), fluorescence angiography (FA), intravenous fluorescein angiography (IVFA), and fundus autofluorescence (FAF). Imaging such as computerized tomography (CT) or magnetic resonance imaging (MRI) scans are utilized to evaluate ocular, periocular and orbital structures, and the intracranial portion of the optic nerve, visual pathway and visual cortex in the brain. These tests allow visualization of structural integrity and thickness of the layers of the eye and surrounding structures, and assessment of blood flow and circulation. Advanced functional testing of the retina, optic nerve and visual pathway/cortex is also used, including electrophysiologic tests such as full field and multifocal electroretinography, visual evoked potentials and microperimetry to diagnose and monitor disease progression and impact of therapy. Those of skill in the art will be able to deploy the appropriate methodologies known in the art to diagnose, measure and monitor the retinal vasculopathies described herein.

Patient: In some embodiments of the claimed invention, the candidate agent inhibitors or agonists of the invention used to treat a patient suffering from DME, is administered to a patient suffering not only from DME but also with diabetic retinopathy (DR). In other embodiments, the DME patient is suffering not only from DME, but also from nonproliferative DR. In other embodiments, the DME patient is also suffering from proliferative DR. In other embodiments of the invention, the patient is suffering from age-related macular degeneration (AMD). In other embodiments, the patient who suffers from AMD, suffers from neovascular AMD.

Administration: Intravitreal (IVT) is one exemplary route of administration of the agents described and claimed herein, in a procedure to place a medication directly into the space in the back of the eye called the vitreous cavity, which is filled with a jelly-like fluid called the vitreous humor gel. The procedure is usually performed by a trained retina specialist in the office setting. Another route of administration of the agents described and claimed herein is intracameral (IC). An intracameral injection is usually into the anterior chamber of the eyeball. Other routes of administration of the agents described and claimed herein may be intravenous, intramuscular, oral, parenteral, topical and subcutaneous.

### EXAMPLES

The following are examples of methods and compositions of the invention. It is understood that various other embodiments may be practiced, given the general description provided above.

### Example 1: Testing Candidate Agents on Senescent Retinal Vascular Cells or Epithelial Cells or Endothelial Cells

The ability of candidate agents to eliminate senescent retinal vascular endothelial cells or senescent retinal pigmented epithelial cells can be measured directly in the following assay. Human retinal microvascular endothelial cells (HRMEC) can be obtained from Neuromics® with the designation HEC09 or Cell Systems ACBRI 181. Human umbilical vein endothelial cells (HUVEC) can be obtained from Lonza® with the designation CC-2519. The cells can be maintained and propagated at <75% confluency in ENDO-Growth® Media (Neuromics®, Edina M N) at 3% O₂, 10% CO₂, and ˜95% humidity. Human retinal pigmented epithelial cells (RPE) can be obtained from Lonza® with the designation 194987. The RPE cells can be maintained and propagated at <75% confluency in RPE media defined by Sonoda et al. in 2009 with 5% FBS and Pen/Strep in an atmosphere of 3% O₂, 10% CO₂, and ˜95% humidity.

The cells can be divided into three groups: irradiated cells (cultured for 7 days after irradiation prior to use), proliferating normal cells (cultured at low density for one day prior to use), and quiescent cells (cultured to confluency over 4 days).

On day 0, the irradiated HRMEC or RPE or HUVEC cells can be incubated with TrypLE trypsin-containing reagent (Thermofisher® Scientific, Waltham, Massachusetts) until the cells round up and begin to detach from the plate. Cells can then be dispersed, counted, and prepared in medium at a concentration of 100,000 cells per mL. This cell suspension can be placed in T175 flasks at a density of 100,000 cells per mL and irradiated at 10-15 Gy. Following irradiation, the cells can be plated at 100 μL in 96-well plates. On days 1, 3, 4, or 6, the medium in each well can be aspirated and replaced with fresh medium. On day 3, the quiescent healthy non-senescent HRMEC or RPE cells can be trypsinized from the culture flask as described above, and the cells can be dispersed, counted, and prepared in medium at a concentration of 80,000 cells per mL. 100 μL of the cells can be plated in each well of a 96-well plate and medium can be changed on days 1, 3, 6 and 10.

On day 10, candidate agents are appropriately diluted to the desired concentration, and then can be combined with the cells, after aspirating off the medium. The candidate agents can be cultured with the cells for an appropriate number of days, such as, for example, 3-7 days. The assay system may use the properties of a thermostable luciferase to enable reaction conditions that generate a stable luminescent signal while simultaneously inhibiting endogenous ATPase released during cell lysis. At the end of the culture period, the plates can be removed from the incubator and allowed to equilibrate at room temperature for 20 minutes, then 100 μL of CellTiter-Glo® reagent (Promega® Corp., Madison, Wisconsin) can be added to each of the wells. The cell plates can be placed for 30 seconds on an orbital shaker and then allowed to stand at room temperature for 10 minutes before measuring luminescence using, for example, an EnVision® plate reader (Perkin Elmer). The luminescence readings can be normalized to determine % cell survival/growth and plotted against candidate agent concentrations and control, and potencies (IC50 values) can be determined by non-linear curve fitting in Graphpad® Prism.

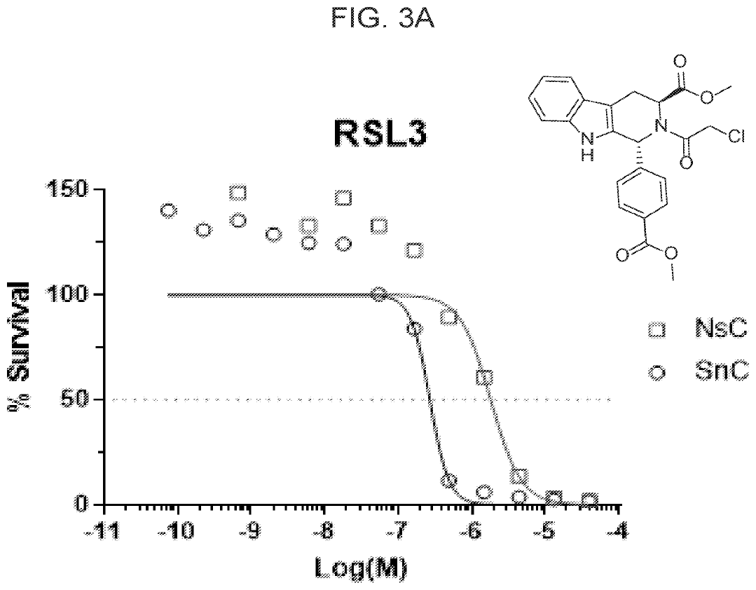

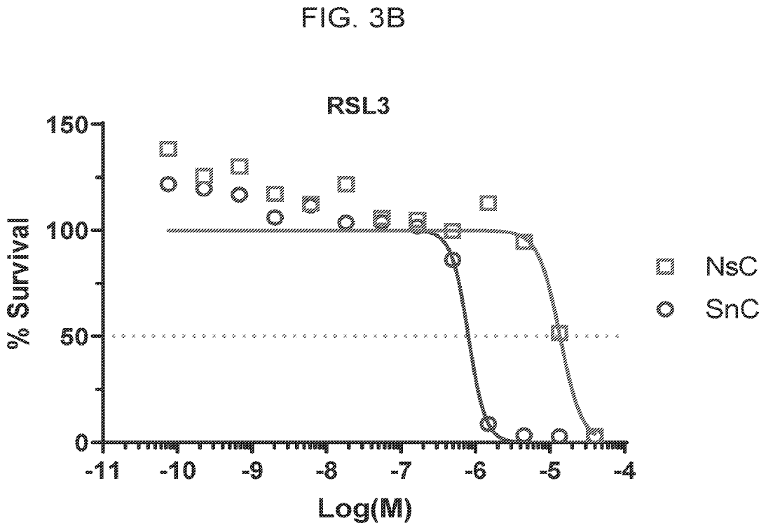

This experiment was performed testing a GPX4 inhibitor, RSL3 ((1S,3R)-methyl 2-(2-chloroacetyl)-1-(4-(methoxycarbonyl)phenyl)-2,3,4,9-tetrahydro-1H-pyrido[3,4-b]indole-3-carboxylate; CAS No. 1219810-16-8), prepared in a one to three dilution series in DMSO at 200× then diluted to 1.5× final concentration in media, and applied on irradiated, senescent HRMEC cells and non-senescent HRMEC cells as a control, see FIG. 3A. The data shows that RSL3 is selectively senolytic on senescent HRMEC cells as compared to non-senescent HRMECs. Similarly, RSL3 was applied on irradiated, senescent HUVEC cells and non-senescent HUVEC cells as a control, see FIG. 3B. The data shows that RSL3 is selectively senolytic on senescent HUVEC cells as compared to non-senescent HUVECs.

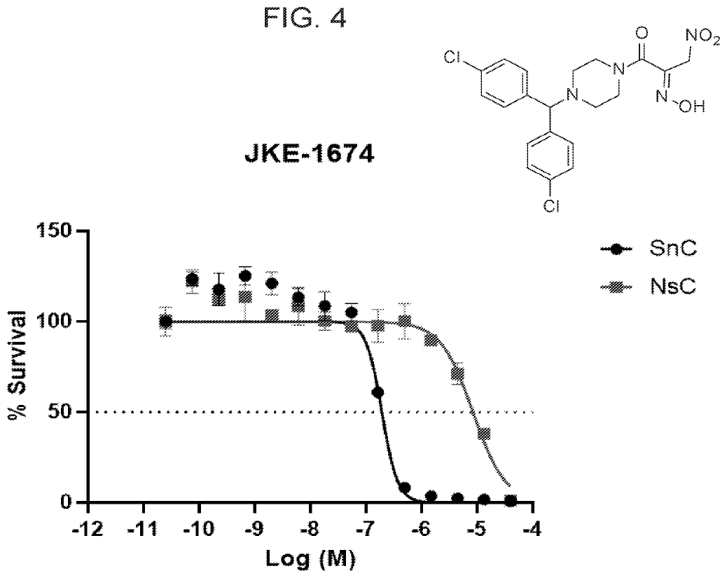

This experiment was also performed testing a different GPX4 inhibitor, JKE-1674 ((E)-1-(4-(bis(4-chlorophenyl)methyl)piperazin-1-yl)-2-(hydroxyimino)-3-nitropropan-1-one; CAS No. 2421119-60-8), prepared in a one to three dilution series in DMSO at 200× then diluted to 1.5× final concentration in media and applied on irradiated, senescent HUVEC cells and non-senescent HUVEC cells as a control, see FIG. 4. The data shows that JKE-1674 is selectively senolytic on senescent HUVEC cells as compared to non-senescent HUVECs.

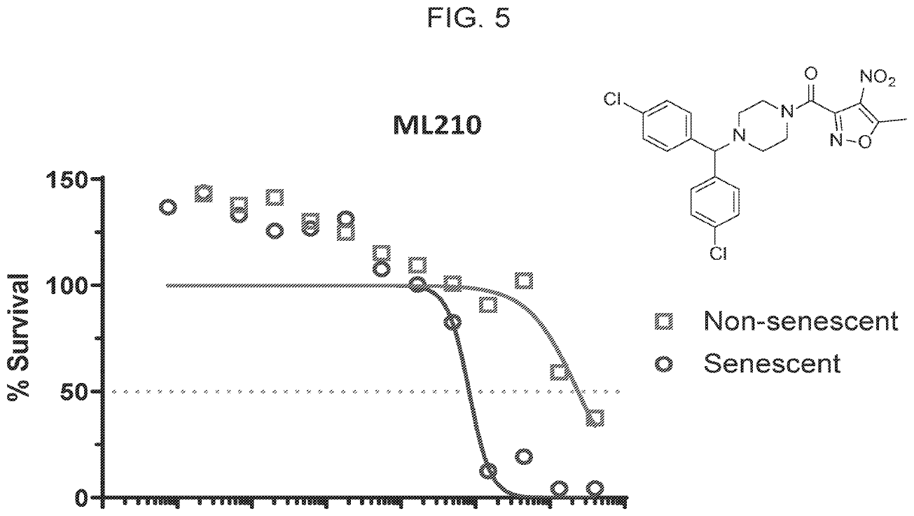

This experiment was also performed testing yet another GPX4 inhibitor, ML210 (see Eaton et al., BioRxiv, Sep. 5, 2018; CAS No. 1360705-96-9), which was prepared in a one to three dilution series in DMSO at 200× then diluted to 1.5× final concentration in media and applied on irradiated, senescent HRMEC cells and non-senescent HRMEC cells as a control, see FIG. 5. The data shows that ML210 is selectively senolytic on senescent HRMEC cells as compared to non-senescent HRMECs.

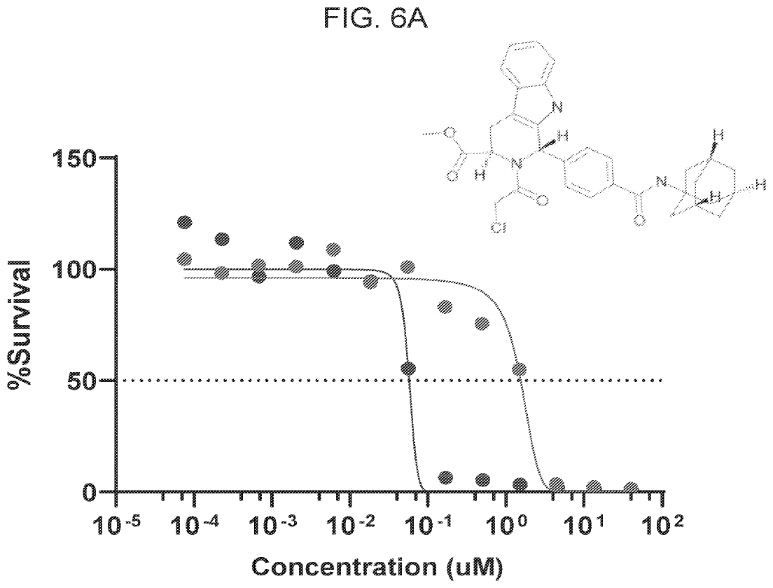

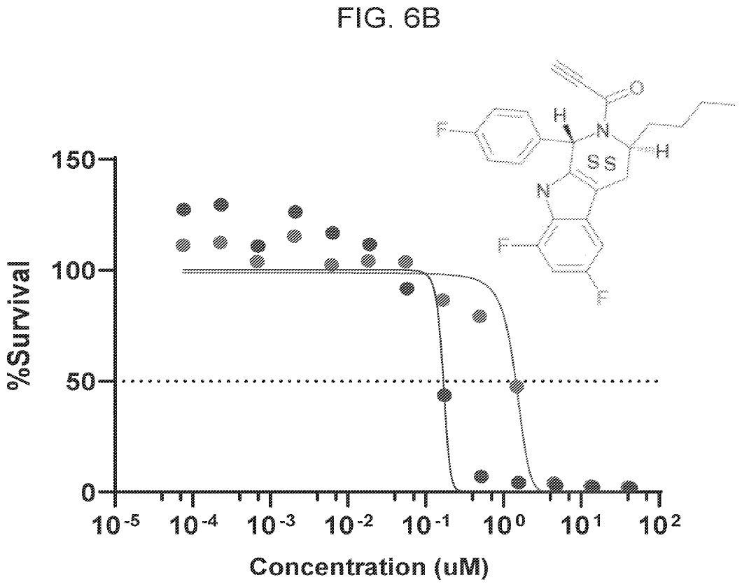

A similar experiment was performed testing two other candidate GPX4 inhibitors, Compounds #19 (see Compound 28 as described in US 2019/0263802) and #25 (see Compound 156 as described in US 2019/0263802), prepared in a one to three dilution series in DMSO at 200× then diluted to 1.5× final concentration in media and applied on irradiated, senescent HUVEC cells and non-senescent HUVEC cells as a control, see FIGS. 6A and 6B. The data show that both Compounds #19 (FIG. 6A) and #25 (FIG. 6B) are selectively senolytic on senescent HUVEC cells as compared to non-senescent HUVECs.

### Example 2: Evidence for Senescent Cells in AMD, DR/DME and PDR Tissues

To obtain evidence for the presence of senescent cells in AMD, DR/DME, and PDR tissue samples from affected patients were stained for p16.

Human post-mortem whole donor globes were procured prospectively from a variety of eye tissue banks. Patient diagnosis was confirmed using patient history. Following enucleation, globes were placed immediately into Davidson's reagent to fix for at least 48 hours. After fixation, the solution was exchanged for 70% ethanol. Tissue was then placed in histology cassettes and processed for paraffin infiltration and embedding. Paraffin blocks were mounted on a microtome and sectioned to 5-9 μm thickness. Cut tissue sections at 4 mm sagittal sections were placed into a 45° C. water bath, and Superfrost® Plus microscope slides were used to pick up sections, wicking away excess water with a Kimwipe®. Slides were then placed upright to dry for 30 min at room temperature, followed by incubation overnight in a 37° C. incubator. Slides were baked for 15-20 min at 60° C. and then allowed to cool to room temperature. Slides were dewaxed using xylene or xylene substitute (e.g., Histoclear®) for 4 min, repeated for a total of 3 incubations. Slides were then rehydrated by serial incubations in decreasing concentrations of ethanol as follows: twice in 100% ethanol for 5 min each, twice in 90% ethanol for 2 min each, twice in 75% ethanol for 2 min each, twice in 50% ethanol for 2 min each, and then rinsed under water for 4 min.

Antigen retrieval was performed by incubation in acidic sodium citrate buffer at 120° C. for 3 min (or 95° C. for 20 min) using a steamer or pressure cooker. Slides were cooled to room temperature for 15 min following by washing twice in Tris-buffered saline supplemented with 0.1% Triton-X 100 (TBST) for 2 min each. Slides were washed twice again with TBST for 5 min each prior to blocking for 1 h at room temperature in TBST containing 5% normal serum, from the species of origin of the secondary antibody to be used. After blocking, the slides were incubated overnight at 4° C. (or for 2 h at room temperature) with p16 primary mouse anti-human p16^{INK4a} antibody (CINtec clone E6H4, Roche, Cat #705-4793) diluted 1:2 in TBST. P16 was visualized using a diethylaminocoumarin DCC fluorochrome assay on a Roche Ventana Discovery Platform. After full slide scanning using a Zeiss Axioscan® microscope (Zeiss®, Oberkochen, Germany), the number of p16INK4a-positive cells was quantified using Visiopharm image analysis software (Visiopharm®, Hoersholm, Denmark), and total cells were quantified from total DAPI stained nuclei, where p<0.0001 v. normal by Kruskal-Wallis with Dunn's multiple comparisons test.

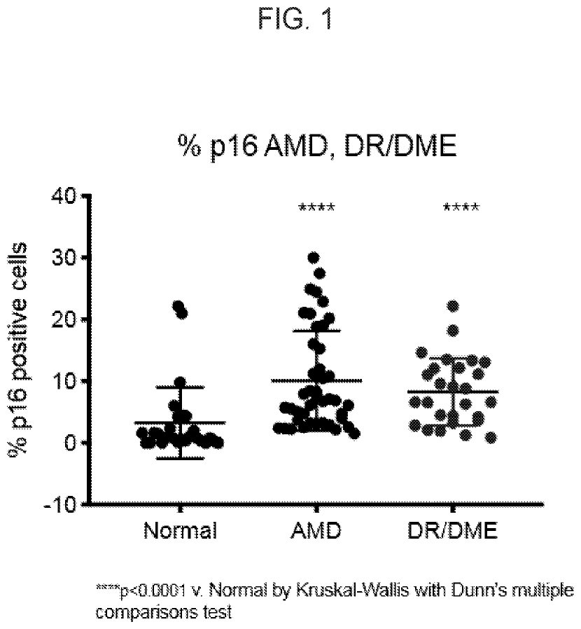

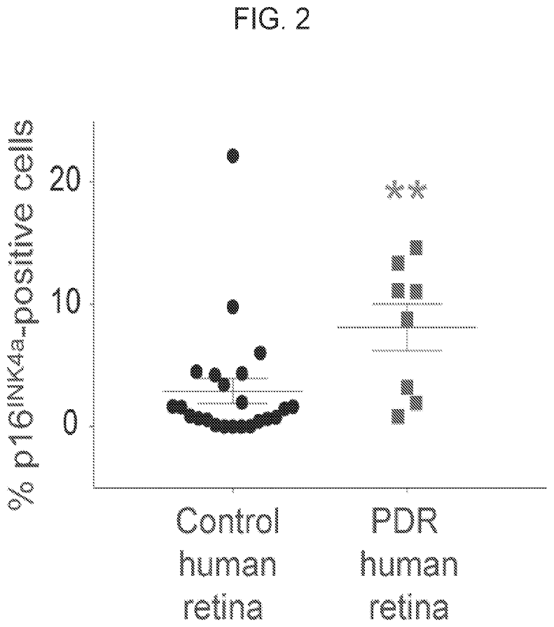

FIGS. 1 and 2 show the quantification of p16^{INK4a}-positive cells show an increase in retinas of patients of AMD, DR/DME and PDR when compared to control retinas.

### Example 3: Efficacy of Candidate Agents in a Bleomycin-Induced Glaucoma Model

This example illustrates the testing of candidate agents of the invention in a mouse model for primary open angle glaucoma (POAG). Male C57BI6/J mice aged 8-10 weeks can be sedated in isofluorane chamber for 3 min then placed on operating table in a nose-cone to maintain constant isofluorane anesthesia. One drop of 2.5% phenylephrine-tropicamide is deposited on the eye for dilation. Measurement of baseline intra-ocular pressure (IOP) can be taken on both eyes using Tonolab™ prior to surgery. The IOP value is reported as an average of six measurements. To induce glaucoma-like phenotype, two μL of bleomycin (0.25 U/kg) or PBS (control) can be intra-camerally injected in the right eye.

IOP measurements can be performed at Day 7 (before treatment), 14, and 21 days after injury. Treatment can be performed 7 days after bleomycin injury. Mice can be sedated in an isofluorane chamber for 3 min then placed on operating table in a nose-cone to maintain constant isofluorane anesthesia. One drop of 2.5% phenylephrine-tropicamide can be deposited on the eye for dilation. Microliter volumes at suitable concentrations of the candidate agents in combination or vehicle only can be intra-camerally injected into one eye.

Eye samples can be collected 14 and 21 days after bleomycin injury. Trabecular meshwork can be collected and fast frozen in liquid nitrogen. Storage of the samples can be at −80° C. until RNA extraction. RNA extraction can be performed using chloroform extraction followed by use of the Direct-Zol Microprep™ RNA extraction kit (VWR®). Five hundred nanograms of RNA can be used to prepare cDNA using the High Capacity Reverse Transcriptase™ kit (ThermoFisher®). One tenth of the cDNA can be used for level of RNA expression measurements using the PerfeCTa qPCR ToughMix Low Rox™ and Taqman™ primer/probe (QuantaBio™).

### Example 4: Efficacy of Candidate Agents in a Diabetes Induced Retinopathy Model

The streptozotocin (STZ) rodent model (Feit-Leichman et al, IOVS 46:4281-87, 2005) recapitulates features of diabetic retinopathy and diabetic macular edema through the induction of hyperglycemia via the direct cytotoxic action of STZ on pancreatic beta cells. Hyperglycemia occurs within days following STZ administration and phenotypic aspects of diabetic retinopathy occur within weeks, with vascular leakage and reduced visual acuity and contrast sensitivity demonstrated in these rodents. This model has thus been widely used for the evaluation of therapeutic agents in diabetic eye disease.

C57BL/6J mice of 6- to 7-weeks are weighed and their baseline glycemia are measured (Accu-Chek®, Roche). Mice can be injected intraperitoneally with STZ (Sigma-Aldrich®, St. Louis, MO) for 5 consecutive days at 55 mg/Kg. Age-matched controls can be injected with buffer only. Glycemia can be measured again a week after the last STZ injection and mice are considered diabetic if their non-fasted glycemia is higher than 17 mM (300 mg/dL). STZ treated diabetic C57BL/6J mice can be intravitreally injected with microliter volumes of candidate agents at 8 and 9 weeks after STZ administration. Retinal vasopermeability can be measured by an Evans blue permeation assay at 10 weeks after STZ treatment as follows.

Mice are anesthetized and injected by tail vein with Evans blue dye dissolved in saline. Two hours after tail vein injection, mice are anesthetized with ketamine and xylazine and can be perfused through the left ventricle using saline. After perfusion, retinas are dissected, weighed and placed in formamide for 18 hours at 70° C. to extract Evans blue dye. On the next day, retinas are centrifuged for 45 minutes and removed from the formamide. Extravasation of Evans blue is measured using a plate reader at A620. A standard curve is used to convert to units of ng Evans blue/wet tissue weight.

### Example 5: Efficacy of Candidate Agents in a Laser-Induced Choroidal Neovascularization (CNV) Mouse Model

The laser-induced CNV model involves rupture of Bruch's membrane, leading to an inflammatory/wound-healing response and concomitant CNV, thereby mimicking wet or neovascular AMD. The choroidal capillaries are explicitly involved in the neovascular response, and this model produces a similar angiographic appearance to wet or neovascular AMD.

Male C57BL/6J mice (6-8 week) can be anesthetized with a ketamine/xylazine cocktail before laser treatment. CNV lesions are induced by laser photocoagulation using a diode laser (IRIDEX®, Oculight® GL) and a slit lamp (Zeiss®) with a spot size of 50 um, power of 180 mW and exposure duration of 100 ms. Four laser burns are typically induced at 3, 6, 9 and 12 o'clock position around the optic disc in each eye. Candidate agents and appropriate controls are injected intraperitoneally one day before laser induction and a total of 3 injections can be performed every 3 days. Nine days after laser induction, mice can be perfused with FITC-lectin or TRITC-dextran via tail vein. After perfusion, the eyes are enucleated and fixed in 4% paraformaldehyde (PFA) for 15 min.

Choroid-sclera complexes and retinas can be separated and anti-CD31 immunofluorescence (IF) can be performed to evidence the vasculature by whole mount staining of both retina and choroidal tissues. For CD31 IF, rat anti-mouse antibody BD 550274 was diluted 1:100 and incubated overnight at 4° C. After 4-hour incubation with a secondary anti-rat antibody (Life Technologies®, A11006) whole mounts can be imaged at 488 nm. Quantification of neovascularization in lesion area and vascular density in retina can be carried out by ImageJ, an open source software developed by the National Institutes of Health. P values can be assessed by Student's t test (significant change, p<0.05).

### Example 6: Efficacy of Candidate Agents in an Oxygen-Induced Retinopathy (OIR) Mouse Model

The OIR model is based on the exposure of mouse pups to hyperoxia during a phase when their retinal vasculature is still developing. This leads to capillary depletion, and upon return to room air, results in retinal ischemia and proliferative vascular disease in the retinal vasculature or oxygen-induced retinopathy.

C57BL/6J pups at postnatal day 7 (P7) are housed in a hyperoxic chamber (75% O2) for 5 days (n=10 per cage) leading to vessel regression in the center of the retina. CD-1 fostering mothers are rotated before and 2-3 days after entering the chamber. At P12, pups are returned to room air where the relative hypoxia triggers abnormal neovascularization, then the appropriate amounts of a control and candidate agent are dosed intraperitoneally. At P17, all groups, including naïve OIR mice can be euthanized. Eyes are enucleated and fixed in 4% paraformaldehyde for 1 hour.

Retinas are dissected and incubated overnight with rhodamine-labeled lectin from Bandeiraea simplicifolia (Griffonia simplicifolia) (1:100) in 1 mM CaCl₂) in PBS to visualize vaso-obliterated (VO) or neovascular (NV) areas. Stained retinas are flat mounted onto slides and imaged on the Zeiss® AxioScan. Images can be analyzed on Visiopharm® to determine % VO or % NV of total retina.

### Example 7: GPX4 Competitive Target Engagement Gel-Shift Assay

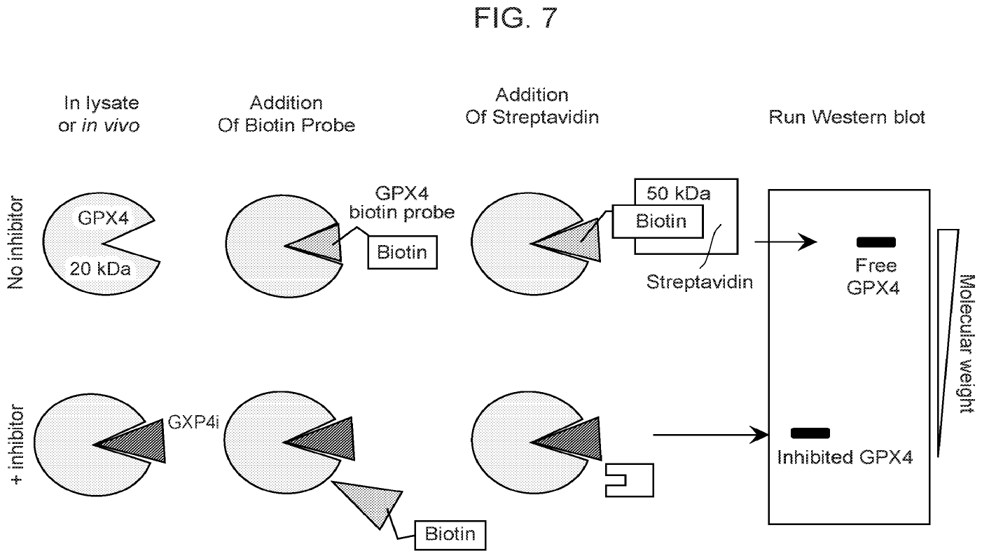

A gel shift assay was developed for the detection of either in vitro cell line or in vivo tissue target engagement (TE) of a GPX4 inhibitor. See FIG. 7 for a schematic of the assay.

In vitro labeling protocol: Two frozen murine retinas were added directly to 0.5 mL Precellys® tubes with ceramic beads and 200 μL of cold mPER with 1× Roche EDTA-free protease inhibitors and 5 mM TCEP. The retina tissue was homogenized using a single 15 s burst at 4500 RPM at 4° C. The tubes were spun at 5 k×g for 5 min @ 4° C. to remove the foam, then all liquid was transferred to 1.5 mL Eppendorfs®. The samples were spun @ 20 k×g for 10 min at 4° C., and the clarified lysates were transferred to clean tubes. 27.5 μL of each of the vehicle controls were combined into one tube to provide a labeling standard sample.

For the labeling control, 50 μL of sample was added to 1 μL of 5 mM RSL3 and another 50 μL of sample was added to DMSO as a control. The samples were mixed and incubated for 1 hr at room temperature (RT). After this 1 μL of a 5 mM GPX4 biotin probe was added to each sample and then were briefly vortexed and incubated for an additional 1 hr 20 min at RT. 300 μL of −20° C. acetone was added at the end of the incubation and the samples were stored at −20° C. For all other samples, 50 μL of the lysate was added to 1 μL of 5 mM GPX4 biotin probe and mixed. The reactions were allowed to proceed at RT for 2 hr before acetone precipitation as above.

Samples were thawed and spun for 10 min at 20,000×g at 4° C. The supernatant was aspirated and discarded. 500 μL ice cold acetone was added to each tube and pellets were broken up with a brief sonication in the bath sonicator. Following this, tubes were spun at 20,000×g and 4° C. for 5 min, rotated 180 degrees and spun for another 5 min. Supernatant was aspirated and the pellets were allowed to air-dry ˜10 min.

Ten μL of SDS solution (0.5% SDS, 150 mM NaCl, 50 mM Tris 7.4, 5 mM TCEP, 1× Roche protease inhibitors) was added to each tube and allowed to stand 1 hr at RT. Tubes were then individually sonicated at a focal point of a bath sonicator, then vortexed and spun briefly to consolidate sample at the bottom of the tube. 40 μL NP40 solution (1.25% NP-40, 150 mM NaCl, 50 mM Tris 7.4, 5 mM TCEP, 1× Roche protease inhibitors) was added and samples were again briefly sonicated, vortexed, and collected at the bottom of the tube.

For the streptavidin binding reaction, 20 μL of the sample was added to 5 μL 5 mg/mL streptavidin (Promega®). A second 20 μL of each reaction was added to a “mock” tube containing 5 μL of 150 mM NaCl. These two 25 μL samples were shaken overnight at 4° C. at 550 RPM.

Western blot analysis: Samples were diluted with 25 μL 2× NuPAGE® sample buffer (diluted from 4×w dH₂O) and loaded onto 4-12% NuPAGE Midi, 1 mm, 12+2 well Bis/Tris gels (Invitrogen). Gels were eluted at 150V for 1 hr 15 m, then transferred using the iBlot® (ThermoFisher) to a nitrocellulose membrane using the Program 3 setting for 7 min.

Blots were blocked with 5% Milk in TBST for 1 hr. After washing with TBST, an anti-GPX4 antibody (Abcam #ab125066), diluted 1:2000 in 5% BSA in TBST, was added and blots were incubated overnight at 4° C. with shaking. Anti-rabbit-HRP was used at 1:5000 in 5% milk in TBST for ˜1 hr RT and signal was detected using the Western Pico® ECL reagent. All analysis were performed using the 5 minute exposures from an Azure Biosystems® c500. The integrated density of bands for +/−streptavidin was quantitated using ImageJ® (National Institutes of Health).

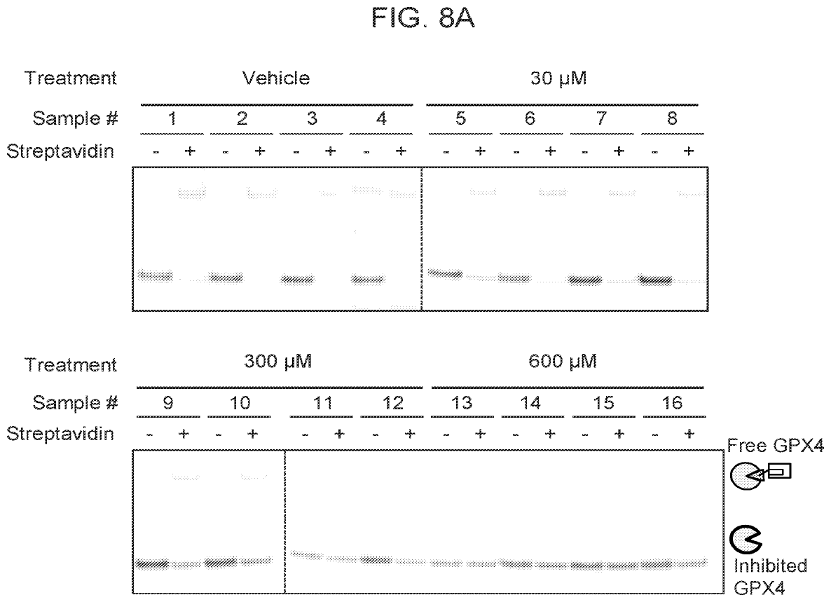

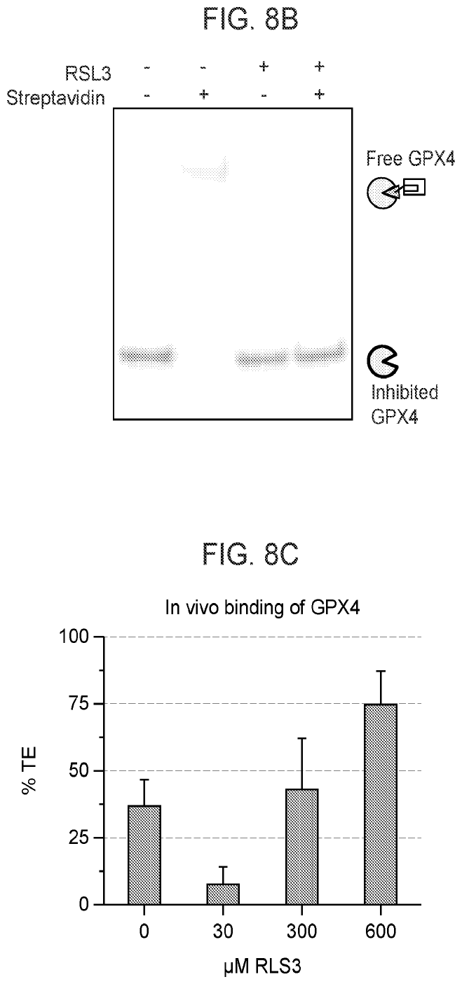

This experiment demonstrated a dose-dependent GPX4 TE for RSL3 in adult murine retinas, see FIGS. 8A-8C.

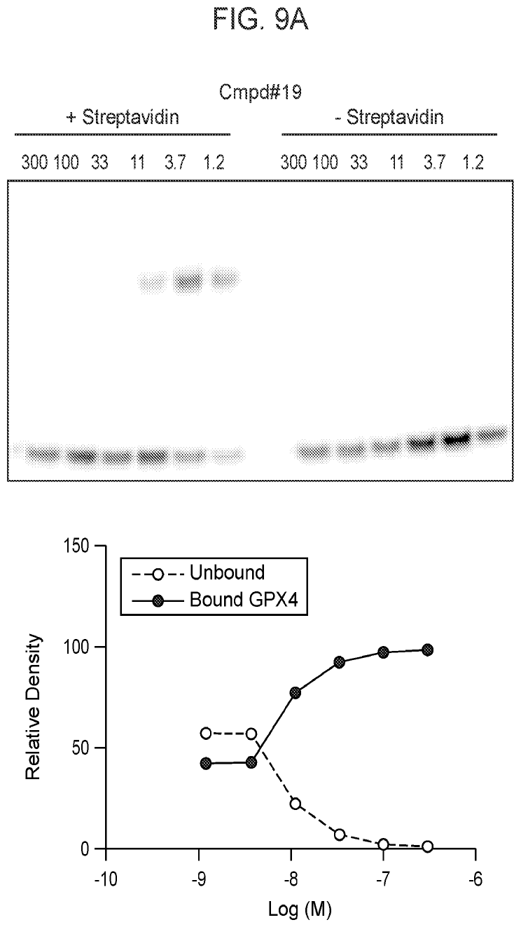

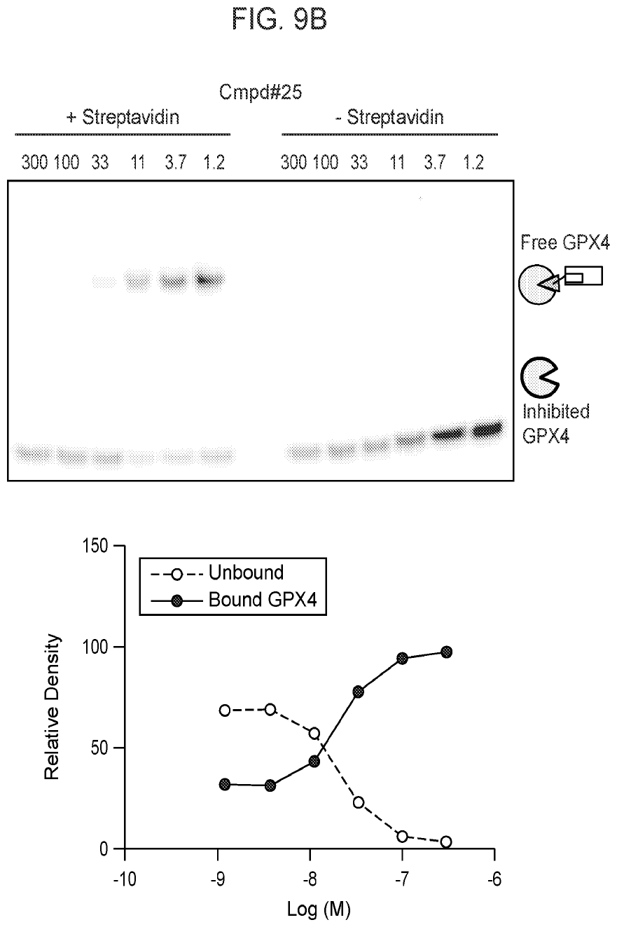

This experiment was also performed using the GPX4 inhibitors Compounds #19 and #25, see FIGS. 9A and 9B respectively, by incubation with senescent HUVEC cells for 6 hours. The data showed potent sub-micromolar in vitro TE.

This gel-shift assay can be used in tandem with the OIR model, described above, to test TE of candidate GPX4 inhibitors in an in vivo situation. Specifically, once abnormal neovascularization is triggered in murine pups, then the appropriate amounts of a vehicle control or a candidate GPX4 inhibitor can be dosed, such as, for example, intravitreally (IVT). After a period of time, the mice are euthanized and the eyes can be collected and processed in the gel-shift assay described herein.

### Example 8: Determination of GCN2 In Vitro Activity

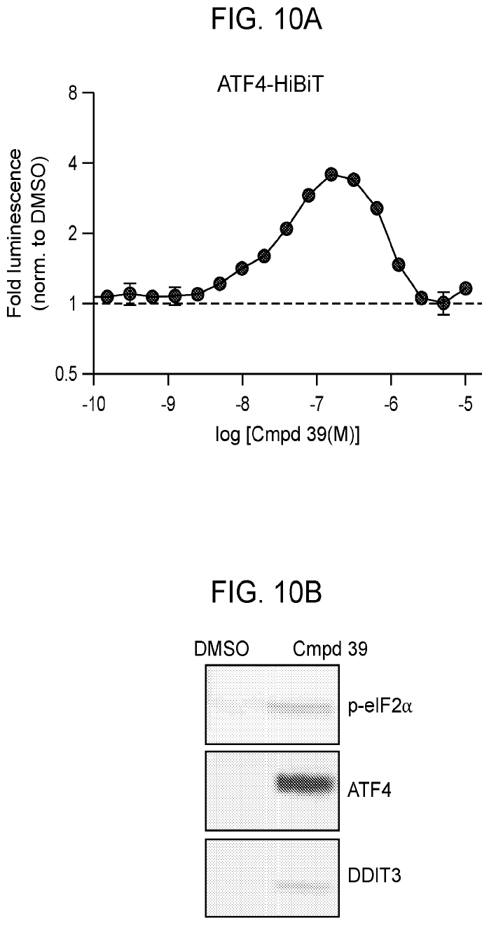

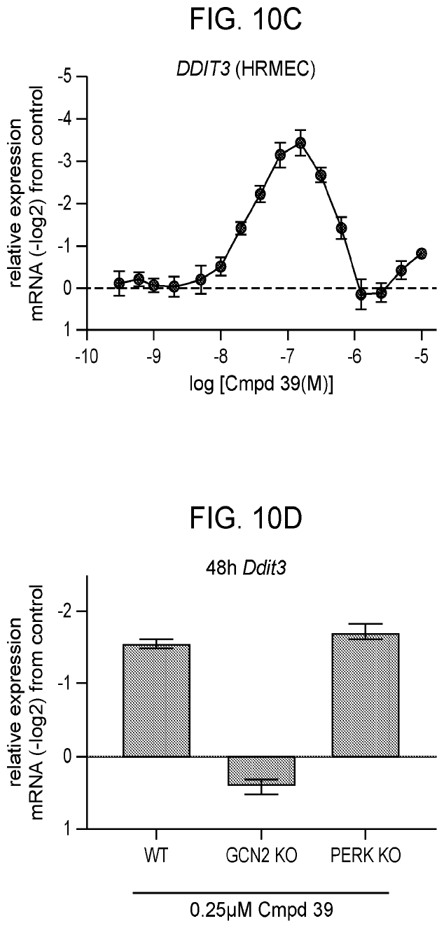

A particular compound, Compound 39, which was originally described as a PERK inhibitor with off-target GCN2 inhibitor activity (see Smith, Adrian L., et al., J of Med Chem. 2015 Feb. 12:58(3):1426-41), was tested to determine whether it possessed GCN2 activation activity using a variety of in vitro assays. Without any limitations, such assays may be used to test other compounds for GCN2 pathway activation. Activating transcription factor 4 (ATF4) was C-terminally tagged in H2122 cells (CRL-5985™) with a high affinity luciferase complementation fragment (HiBiT) through a knock-in approach using CRISPR-Cas12a technology and a single-stranded oligo DNA nucleotides (ssODNs) template carrying the HiBiT tag. Following reporter cell-line generation (H2122 ATF4-HiBIT) cells were seeded in a 96-well plate at near confluency and treated the next day with Compound 39 for five hours. A dose response was generated using quarter-log dilutions using 10 μM as the starting concentration. The Nano-Glo® HiBiT Lytic Detection (Promega®) reagent was added after five hours and allowed for plate-based bioluminescent quantification of cellular ATF4 protein using an add-mix-read assay format. Peak activation of ATF4 expression was detected at ˜250 nM of Compound 39. See FIG. 10A.

Next, mouse embryonic fibroblasts (MEF) cells were cultured to near confluency and treated for five hours with a fixed concentration (250 nM) of Compound 39. Cells were harvested in LSD lysis buffer and homogenates were separated by SDS-PAGE. Immunoblotting was performed overnight using antibodies targeting p-eIF2a (Clone D9G8), ATF4 (Clone D4B8), and DNA damage inducible transcript 3 (DDIT3) (Clone 9C8). Compound 39 treatment increases the levels of phospho-eIF2a (p-eIF2a) as well as ATF4 and DDIT3 (CHOP). See FIG. 10B.

Primary Human Retinal Microvascular Endothelial Cells (HRMEC—Cell Systems™ Corp., ACBRI 181; Lot #181.04.02.02.02) were cultured in Vascular Cell Basal Medium (ATCC® Lot 90909277) with Endothelial Cell Growth Kit-VEGF (ATCC Lot 80720201) with additional FBS added to a final concentration of 5% FBS. Cells were seeded in a 96-well plate at near confluency and treated the next day with Compound 39 for five hours. A dose response was generated using quarter-log dilutions using 10 μM as the starting concentration. Following treatment, RNA was extracted using the RNeasy® 96-Kit (Qiagen®), and cDNA was next generated using Superscript™ IV VILO™ Master Mix (Thermofisher®). qPCR was performed using standard TaqMan® reagents and conditions. DDTI3 gene expression was detected using primer Hs00358796_g1 and Tbp was used as a control gene using primer (Hs99999910_m1). Peak activation of DDIT3 expression was detected at ˜250 nM Compound 39. See FIG. 10C.

To determine if DDIT3 activation by Compound 39 is dependent on GCN2 activation, rather than PERK, the following experiment was performed. GCN2 knockout cells (CRL-2978™), PERK knockout cells (CRL-2976™), or WT controls cells (ATCC CRL-2977™) were cultured to near confluency and treated for five hours with a fixed concentration (250 nM) of Compound 39. Following treatment, RNA was extracted using the RNeasy® 96-Kit (Qiagen), and cDNA was next generated using Superscript™ IV VILO™ Master Mix (Thermofisher®). qPCR was performed using standard TaqMan reagents and conditions. DDIT3 gene expression was detected using primer Hs00358796_g1 and Tbp was used as a control gene using primer (Hs99999910_m1). Knockout cell-lines indicated that DDIT3 induction by Compound 39 was dependent on GCN2 but not PERK. See FIG. 10D.

### Example 9: Determination of GCN2 In Vivo Activity

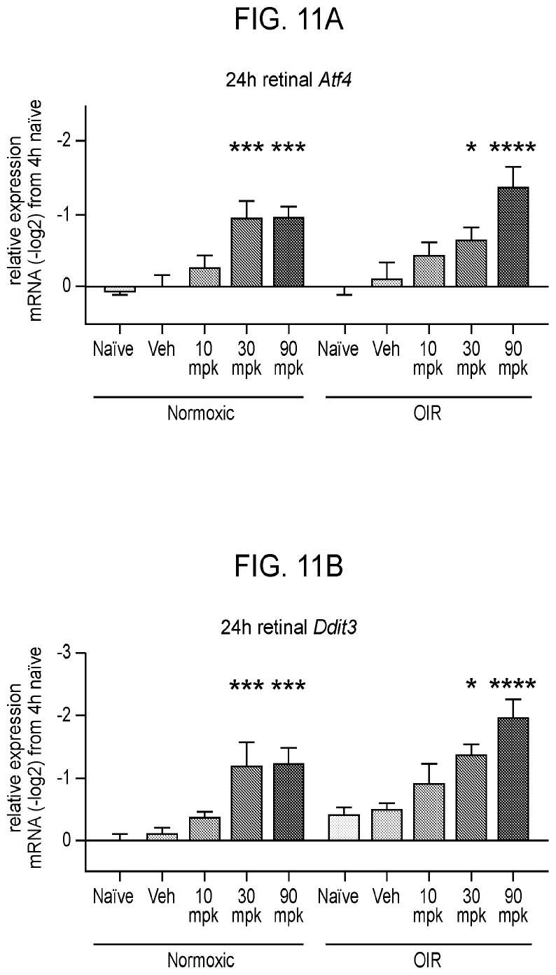

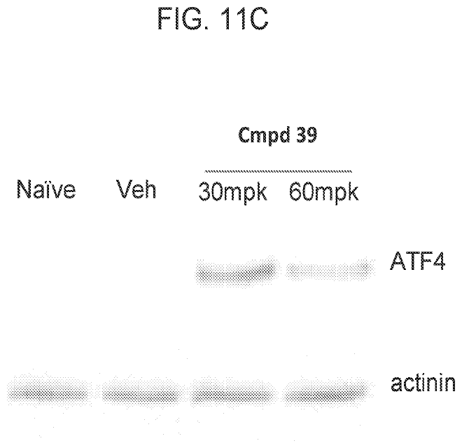

Compound 39 was tested in an animal model that shares many hallmarks with human ischemic retinopathies, the oxygen-induced retinopathy (OIR) neonatal mouse. To investigate the pharmacodynamic effect of Compound 39, it was intraperitoneally (IP) administered at either 10, 30, or 90 mpk. The dosing formulation was based on a vehicle comprising 1% Pluronic F-68/1% HPMC, 15% captisol. Both normoxic and OIR neonates were treated at P12 (the end of hyperoxia) and taken down after 4 hours, or 24 hours later at P13. Retinas were dissected, and RNA was extracted using a fully automated, spin-column-based nucleic acid extraction instrument (Qiacube Connect™). qPCR was performed using standard TaqMan® reagents and conditions. Atf4, Ddit3, and Tbp (control) gene expression was determined using primers: Mm00515324_m1, Mm00492097_m1, and Mm01277042_m1, respectively. Compound 39 was shown to have a dose-dependent induction of gene expression for both ATF4 (FIG. 11A) and DDIT3 (FIG. 11B) following IP administration.

Intraperitoneally administered Compound 39 activated ATF4 protein expression at 30 and 60 mpk. Adult eye retinas were dissected 4 hours post Compound 39 administration and the harvested samples were kept on ice and homogenized using Precellys™ (500 μL tubes) tissue homogenizer in 300 μL MSD Tris Lysis buffer containing Pierce Protease Inhibitor Complete Mini tablet (ThermoScientific™ A32963). Homogenized samples were then centrifuged to separate supernatant from debris. Lysates were separated by SDS-PAGE and immunoblotted for ATF4 (D4B8) and control protein Actinin (Cell Signaling, cat. 3134S). See FIG. 11C. The results demonstrate that Compound 39 increased the levels of ATF4 but not control protein Actinin.

### Example 10: Modulation of Stress Response Genes with Relevance to Retinal Diseases Using a GCN2 Agonist

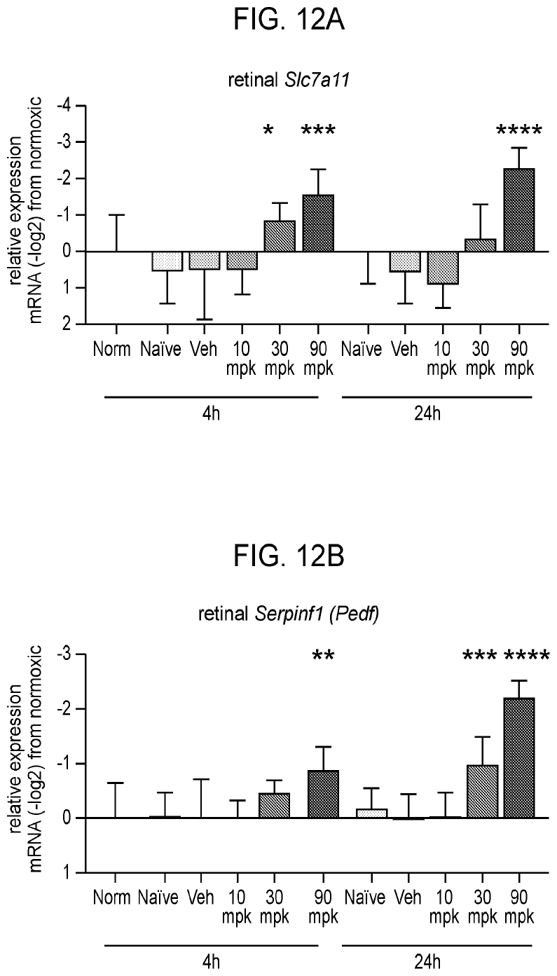

To investigate the pharmacodynamic effect of Compound 39, it was intraperitoneally administrated at either 10, 30, or 90 mpk in both normoxic and OIR neonatal mice. The dosing formulation was based on a vehicle comprising 1% Pluronic F-68/1% HPMC, 15% captisol. Both normoxic and OIR neonates were treated at P12 (the end of hyperoxia) and taken down after 4 hours, or 24 hours later at P13. Retinas were dissected, and RNA was extracted using a fully automated, spin-column-based nucleic acid extraction instrument (Qiacube Connect™). qPCR was performed using standard TaqMan reagents and conditions. Slc7a11, Serpinf1, and Tbp (control) gene expression was determined using primers Mm00442530_m1, Mm00441270_m1, and Mm01277042_m1, respectively. A dose-dependent induction of gene expression by Compound 39 was detected for both retinal Slc7a11 (FIG. 12A) and retinal Serpinf1 (FIG. 12B).

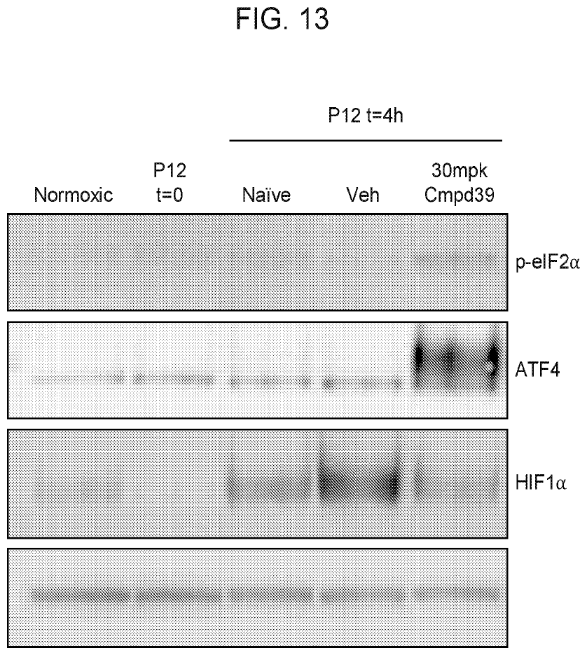

Normoxic and OIR eye retinas were dissected 4 hours after Compound 39 administration and the harvested samples were kept on ice and homogenized using Precellys (500 μL tubes) tissue homogenizer in 300 μL LSD Sample/Lysis buffer. Homogenized samples were then centrifuged to separate supernatant from debris. Lysates were separated by SDS-PAGE and immunoblotted for ATF4 (D4B8), p-eIF2a (D9G8), and HIF1α (Novus™, cat. NB100-449) and control protein Actinin (Cell Signaling™, cat. 3134S). See FIG. 13. Compound 39 treatment led to a pharmacodynamic response that featured increased levels of ATF4 and phospho-eIF2α levels, and downregulated HIF1α protein levels-indicating a reduction of hypoxic stress signaling.

### Example 11: Improvement of Retinal Vasculature Using a GCN2 Agonist

The efficacy of Compound 39 was studied in the mouse OIR model. Exposure of young mice to a hyperoxic environment leads to obliteration of retinal vasculature, followed by pathological angiogenesis (neovascularization) upon return to ambient air. Compound 39 was either administered twice intraperitoneally (90 mpk) at the indicated timepoints (P12 and P14 in the model), or just once (at P12, followed bt vehicle control at P14).

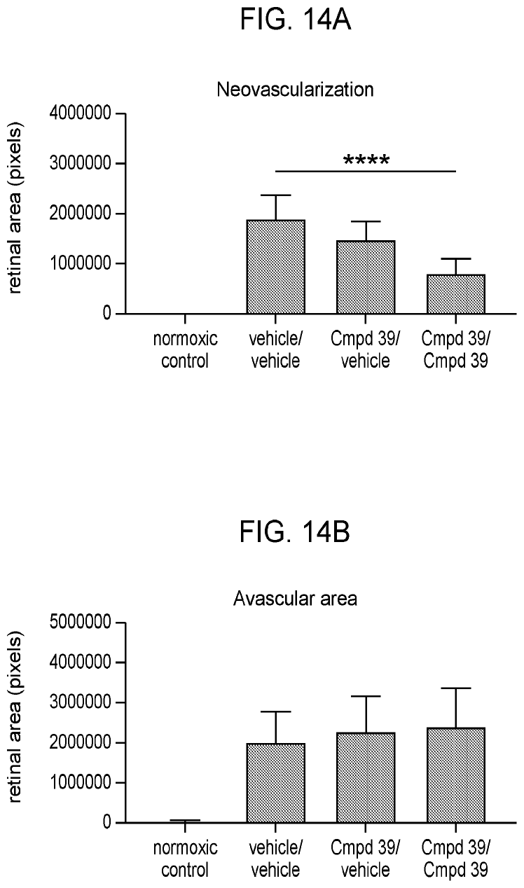

Avascular areas and areas of neovascularization were quantified after harvest and staining of retinal flatmounts at P17 in the model. OIR pups that received two 90 mpk intraperitoneal doses of Compound 39 showed significant reversal of neovascularization (FIG. 14A) but not vaso-obliteration (FIG. 14B) at P17. These results show that a systemically administered Compound 39 can functionally inhibit pathogenic angiogenesis in the OIR disease model.

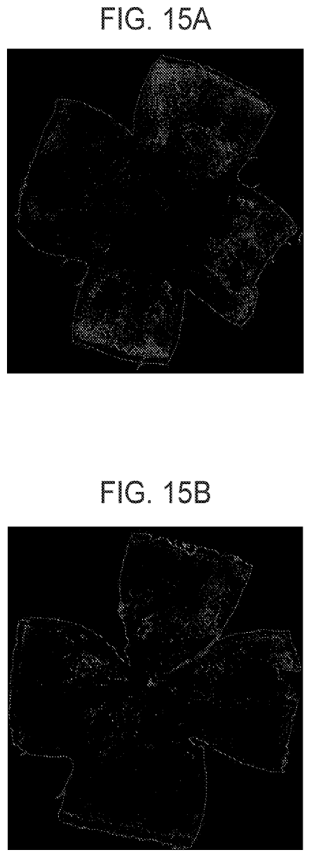

OIR pups were treated with intraperitoneal injection of 90 mpk Compound 39 at P12, then these pups were removed to room air with their nursing mothers, repeat dosed at P14, and were sacrificed at P17. Eyes were enucleated at P17 and retinas dissected for vascular staining. To determine avascular or neovascular areas, retinas were flatmounted, and stained with isolectin B4 (IB4) for fluorescence microscopy. The isolectin-stained flatmounts show the preservation of retinal vessels in a pup treated with Compound 39 (FIG. 15B) compared to a pup treated with vehicle (FIG. 15A). The vehicle-treated pup has an ischemic, posterior retina compared to the repeat Compound 39-treated pup indicating that two intraperitoneal administrations of 90 mpk of Compound 39 significantly suppressed neovascularization.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of inhibiting pathogenic neovascularization in a diseased eye comprising administering an agent, wherein the agent is selected from the group consisting of an inhibitor of GPX4, an inhibitor of GLS1, an inhibitor of PAPP-A, an inhibitor of cGAS, an inhibitor of STING, an inhibitor of mTOR, or an agonist of GCN2.

2. A method of inhibiting vascular leak in a diseased eye comprising administering an agent, wherein the agent is selected from the group consisting of an inhibitor of GPX4, an inhibitor of GLS1, an inhibitor of PAPP-A, an inhibitor of cGAS, an inhibitor of STING, an inhibitor of mTOR, or an agonist of GCN2.

3. The method of any of claim 1 or 2, wherein the diseased eye is a result of senescent endothelial cells.

4. The method of any of claim 1 or 2, wherein the diseased eye is a result of senescent pericytes.

5. The method of any of claim 1 or 2, wherein the diseased eye is a result of diabetic macular edema (DME), diabetic retinopathy (DR), proliferative diabetic retinopathy (PDR), dry age-related macular degeneration (dAMD), and neovascular age-related macular degeneration (nvAMD).

6. A method of eliminating senescent endothelial cells comprising contacting the cells with an inhibitor of GPX4, an inhibitor of GLS1, an inhibitor of PAPP-A, an inhibitor of cGAS, an inhibitor of STING, an inhibitor of mTOR, or an agonist of GCN2.

7. A method of eliminating senescent pericytes comprising contacting the cells with an inhibitor of GPX4, an inhibitor of GLS1, an inhibitor of PAPP-A, an inhibitor of cGAS, an inhibitor of STING, an inhibitor of mTOR, or an agonist of GCN2.

8. A method of treating a patient suffering from a retinal vasculopathy comprising administering an agent to the patient, wherein the agent is selected from the group consisting of an inhibitor of GPX4, an inhibitor of GLS1, an inhibitor of PAPP-A, an inhibitor of cGAS, an inhibitor of STING, an inhibitor of mTOR, or an agonist of GCN2.

9. The method of claim 8, wherein the retinal vasculopathy is diabetic macular edema (DME), diabetic retinopathy (DR), proliferative diabetic retinopathy (PDR), dry age-related macular degeneration (dAMD), and neovascular age-related macular degeneration (nvAMD).

10. The method of any one of claims 1-5, 8-9, wherein the agent is a small molecule, an antibody, a polypeptide, an antisense oligonucleotide, a small-interfering ribonucleic acid.

11. The method of claim 6 or 7, wherein the inhibitor or agonist is a small molecule, an antibody, a polypeptide, an antisense oligonucleotide, a small-interfering ribonucleic acid.

12. The method of any one of claims 1-11, wherein the inhibitor of GPX4 is selected from the group consisting of RSL3, ML210, ML162, JKE-1674, DPI-7, buthionine sulfoximine (BSO), FIN56, auranofin, erastin, artemisinin, sulfasalazine, artesunate, dihydroatemisin, Compound #19, Compound #25, sorafenib, altretamine, almitrine, artemether, artemisone, lanperisone.

13. The method of any one of claims 1-11, wherein the inhibitor of GLS1 is 6-Diazo-5-oxo-L-norleucine, GK921, UPGL00004, telaglenastat, JHU395, Ethyl 2-(2-Amino-4-methylpentanamido)-DON, IPN60090, and BPTES.

14. The method of any one of claims 1-11, wherein the inhibitor of PAPP-A is an antibody.

15. The method of claim 14, wherein the antibody is PAC-1 scFV, PAC-1-D8 scFv, PAC-2 scFv, PAC-5 scFv, and mAb-PA 1/41.

16. The method of any one of claims 1-11, wherein the inhibitor of PAPP-A is a polypeptide.

17. The method of claim 16, wherein the polypeptide is pro-MBP, stanniocalcin-1 (STC1), stanniocalcin-2 (STC2) and bikunin.

18. The method of any one of claims 1-11, wherein the inhibitor of PAPP-A is an antisense oligonucleotide.

19. The method of any one of claims 1-11, wherein the inhibitor of cGAS is 3-(1-(6,7-dichloro-1H-benzo[d]imidazol-2-yl)-5-hydroxy-3-methyl-1H-pyrazol-4-yl)isobenzofuran-1(3H)-one, 1-(6,7-Dichloro-9-(1-methyl-1H-pyrazol-3-yl)-1,3,4,5-tetrahydro-2H-pyrido[4,3-b]indol-2-yl)-2 hydroxyethan-1-one, 1-[9-(6-amino-3-pyridinyl)-6,7-dichloro-1,3,4,5-tetrahydro-2H-pyrido[4,3b]indol-2-yl]-2-hydroxy-ethanone, or (1R,2S)-2-(7-Oxo-5-phenyl-4,7-dihydropyrazolo[1,5-a]pyrimidine-3 carboxamido) cyclohexane-1-carboxylic acid.

20. The method of any one of claims 1-11, wherein the inhibitor of STING is H-151, C-178, C-176, and Compound 18.

21. The method of any one of claims 1-11, wherein the inhibitor of mTOR is rapamycin, Palomid 529, sirolimus, everolimus, temsirolimus, ridaforolimus, umirolimus, zotarolimus and ATP-competitive mTOR kinase inhibitors.

22. The method of any one of claims 1-11, wherein the agonist of GCN2 is 4-(2-amino-4-methyl-3-(2-methylquinolin-6-yl)benzoyl)-1-methyl-2,5-diphenyl-1H-pyrazol-3(2H)-one, 1-(5-(4-amino-2,7-dimethyl-7H-pyrrolo[2,3-d]pyrimidin-5-yl)indolin-1-yl)-2-(3-fluoro-5-(trifluoromethyl)phenyl)-ethanone, 4-(2-Amino-4-methyl-3-(2-(methylamino)benzo[d]thiazol-6-yl)benzoyl)-1-methyl-2,5-diphenyl-1H-pyrazol-3(2H)-one, leucenol, histidinol, threoninol, SB-203207, SB-219383, dovitinib, neratinib, sunitinib, and elotinib.
